SEQUENCE LISTING (110 sequences)

SEQ ID NO: 1 - protein, 5 aa, Artificial sequence
  Synthetic amino acid sequence
TYTMH

SEQ ID NO: 2 - protein, 17 aa, Artificial sequence
  Synthetic amino acid sequence
DIAYDGSTKYYADSVKG

SEQ ID NO: 3 - protein, 11 aa, Artificial sequence
  Synthetic amino acid sequence
DAVAGEGYFDL

SEQ ID NO: 4 - protein, 11 aa, Artificial sequence
  Synthetic amino acid sequence
QGDSLRSYYAS

SEQ ID NO: 5 - protein, 7 aa, Artificial sequence
  Synthetic amino acid sequence
RNNQRPS

SEQ ID NO: 6 - protein, 11 aa, Artificial sequence
  Synthetic amino acid sequence
AAWDDSLSAWV

SEQ ID NO: 7 - protein, 5 aa, Artificial sequence
  Synthetic amino acid sequence
ASGMH

SEQ ID NO: 8 - protein, 17 aa, Artificial sequence
  Synthetic amino acid sequence
FIAYDGNQKFYADSVKG

SEQ ID NO: 9 - protein, 10 aa, Artificial sequence
  Synthetic amino acid sequence
EMQREGYFDY

SEQ ID NO: 10 - protein, 11 aa, Artificial sequence
  Synthetic amino acid sequence
QGDSLRSYYAS

SEQ ID NO: 11 - protein, 7 aa, Artificial sequence
  Synthetic amino acid sequence
GKNNRPS

SEQ ID NO: 12 - protein, 11 aa, Artificial sequence
  Synthetic amino acid sequence
ATWDDNLSGPI

SEQ ID NO: 13 - protein, 5 aa, Artificial sequence
  Synthetic amino acid sequence
DYYMS

SEQ ID NO: 14 - protein, 17 aa, Artificial sequence
  Synthetic amino acid sequence
YISTSGSSIYYVDSVKG

SEQ ID NO: 15 - protein, 10 aa, Artificial sequence
  Synthetic amino acid sequence
DLHGDYAFDS

SEQ ID NO: 16 - protein, 11 aa, Artificial sequence
  Synthetic amino acid sequence
QGDSLRSYYAS

SEQ ID NO: 17 - protein, 7 aa, Artificial sequence
  Synthetic amino acid sequence
GKNNRPS

SEQ ID NO: 18 - protein, 11 aa, Artificial sequence
  Synthetic amino acid sequence
ATWDDNLSGPI

SEQ ID NO: 19 - protein, 5 aa, Artificial sequence
  Synthetic amino acid sequence
SYAMS

SEQ ID NO: 20 - protein, 17 aa, Artificial sequence
  Synthetic amino acid sequence
AISGSGGSTYYADSVKG

SEQ ID NO: 21 - protein, 10 aa, Artificial sequence
  Synthetic amino acid sequence
VSSSWSHFDY

SEQ ID NO: 22 - protein, 11 aa, Artificial sequence
  Synthetic amino acid sequence
RASQYISNWLA

SEQ ID NO: 23 - protein, 7 aa, Artificial sequence
  Synthetic amino acid sequence
KASSLES

SEQ ID NO: 24 - protein, 10 aa, Artificial sequence
  Synthetic amino acid sequence
QESYNTPLFT

SEQ ID NO: 25 - protein, 5 aa, Artificial sequence
  Synthetic amino acid sequence
NYAIN

SEQ ID NO: 26 - protein, 17 aa, Artificial sequence
  Synthetic amino acid sequence
NIHHDGNGKYYVDSVEG

SEQ ID NO: 27 - protein, 9 aa, Artificial sequence
  Synthetic amino acid sequence
DGYGGYLDL

SEQ ID NO: 28 - protein, 11 aa, Artificial sequence
  Synthetic amino acid sequence
QGDSLRSYYAS

SEQ ID NO: 29 - protein, 7 aa, Artificial sequence
  Synthetic amino acid sequence
GKNNRPS

SEQ ID NO: 30 - protein, 11 aa, Artificial sequence
  Synthetic amino acid sequence
AAWDDSLSGPV

SEQ ID NO: 31 - protein, 5 aa, Artificial sequence
  Synthetic amino acid sequence
SYAMH

SEQ ID NO: 32 - protein, 17 aa, Artificial sequence
  Synthetic amino acid sequence
VISYDGSNKYYADSVKG

SEQ ID NO: 33 - protein, 11 aa, Artificial sequence
  Synthetic amino acid sequence
DLSGYGDYPDY

SEQ ID NO: 34 - protein, 11 aa, Artificial sequence
  Synthetic amino acid sequence
QGDSLRSYYAS

SEQ ID NO: 35 - protein, 7 aa, Artificial sequence
  Synthetic amino acid sequence
GRNERPS

SEQ ID NO: 36 - protein, 11 aa, Artificial sequence
  Synthetic amino acid sequence
AGWDDSLTGPV

SEQ ID NO: 37 - protein, 119 aa, Artificial sequence
  Synthetic amino acid sequence
QVQLQESGGGLVQPGGSLRLSCAASGFSFNTYTMHWVRQAPGKGLEWVADIAYDGSTKYYADSVKGRFTISRDNAKNSLYLQMNSLRAEDTAVYYCARDAVAGEGYFDLWGRGTLVTVS

SEQ ID NO: 38 - protein, 111 aa, Artificial sequence
  Synthetic amino acid sequence
SQSALTQDPAVSVALGQTVRITCQGDSLRSYYASWYQQLPGTAPKLLIYRNNQRPSGVPDRFSGSKSGTSASLAISGLRSEDEADYYCAAWDDSLSAWVFGGGTKLTVLGA

SEQ ID NO: 39 - protein, 118 aa, Artificial sequence
  Synthetic amino acid sequence
QVQLQQSGGGVVQPGGSLRLSCAASEFTFSASGMHWVRQAPGKGLEWMAFIAYDGNQKFYADSVKGRFTISRDNSKNTLYLQMDSLRGEDTAVYYCAKEMQREGYFDYWGQGTLVTVS

SEQ ID NO: 40 - protein, 110 aa, Artificial sequence
  Synthetic amino acid sequence
SNFMLTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 41 - protein, 118 aa, Artificial sequence
  Synthetic amino acid sequence
QVQLAESGGGLVKPGGSLRLSCAASGFTFSDYYMSWIRQAPGKGLEWVSYISTSGSSIYYVDSVKGRFTISRDNAKNSLYLQMDSLRDDDTAVYYCARDLHGDYAFDSWGQGTLVTVS

SEQ ID NO: 42 - protein, 109 aa, Artificial sequence
  Synthetic amino acid sequence
SSELTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 43 - protein, 118 aa, Artificial sequence
  Synthetic amino acid sequence
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKVSSSWSHFDYWGQGTLVTVS

SEQ ID NO: 44 - protein, 110 aa, Artificial sequence
  Synthetic amino acid sequence
SDVVMTQSPSTLSASVGDRVTITCRASQYISNWLAWYQQKPGKAPKLLIYKASSLESGVPSRFSGSGSGTEFTLTISSLQPEDFATYYCQESYNTPLFTFGPGTKLEIKR

SEQ ID NO: 45 - protein, 117 aa, Artificial sequence
  Synthetic amino acid sequence
QVQLVESGGGLVEPGGSLRLSCAASGFTFSNYAINWVRQAPGKGLEWVANIHHDGNGKYYVDSVEGRFTISRDNAKNSLYLQMDSLRAEDTAIYYCARDGYGGYLDLWGQGTLVTVS

SEQ ID NO: 46 - protein, 109 aa, Artificial sequence
  Synthetic amino acid sequence
SSELTQDPAVSVALGQTVRITCQGDSLRSYYASWYQQKPGQAPVLVIYGKNNRPSGIPDRFSGSGSGNTASLTITGAQAEDEADYYCAAWDDSLSGPVFGGGTKVTVLG

SEQ ID NO: 47 - protein, 119 aa, Artificial sequence
  Synthetic amino acid sequence
QVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVS

SEQ ID NO: 48 - protein, 109 aa, Artificial sequence
  Synthetic amino acid sequence
SSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 49 - protein, 245 aa, Artificial sequence
  Synthetic amino acid sequence
QVQLQESGGGLVQPGGSLRLSCAASGFSFNTYTMHWVRQAPGKGLEWVADIAYDGSTKYYADSVKGRFTISRDNAKNSLYLQMNSLRAEDTAVYYCARDAVAGEGYFDLWGRGTLVTVSSGGGGSGGGGSGGGGSQSALTQDPAVSVALGQTVRITCQGDSLRSYYASWYQQLPGTAPKLLIYRNNQRPSGVPDRFSGSKSGTSASLAISGLRSEDEADYYCAAWDDSLSAWVFGGGTKLTVLGA

SEQ ID NO: 50 - protein, 243 aa, Artificial sequence
  Synthetic amino acid sequence
QVQLQQSGGGVVQPGGSLRLSCAASEFTFSASGMHWVRQAPGKGLEWMAFIAYDGNQKFYADSVKGRFTISRDNSKNTLYLQMDSLRGEDTAVYYCAKEMQREGYFDYWGQGTLVTVSSGGGGSGGGGSGGGGSNFMLTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 51 - protein, 242 aa, Artificial sequence
  Synthetic amino acid sequence
QVQLAESGGGLVKPGGSLRLSCAASGFTFSDYYMSWIRQAPGKGLEWVSYISTSGSSIYYVDSVKGRFTISRDNAKNSLYLQMDSLRDDDTAVYYCARDLHGDYAFDSWGQGTLVTVSSGGGGSGGGGSGGGGSSELTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 52 - protein, 243 aa, Artificial sequence
  Synthetic amino acid sequence
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKVSSSWSHFDYWGQGTLVTVSSGGGGSGGGGSGGGGSDVVMTQSPSTLSASVGDRVTITCRASQYISNWLAWYQQKPGKAPKLLIYKASSLESGVPSRFSGSGSGTEFTLTISSLQPEDFATYYCQESYNTPLFTFGPGTKLEIKR

SEQ ID NO: 53 - protein, 241 aa, Artificial sequence
  Synthetic amino acid sequence
QVQLVESGGGLVEPGGSLRLSCAASGFTFSNYAINWVRQAPGKGLEWVANIHHDGNGKYYVDSVEGRFTISRDNAKNSLYLQMDSLRAEDTAIYYCARDGYGGYLDLWGQGTLVTVSSGGGGSGGGGSGGGGSSELTQDPAVSVALGQTVRITCQGDSLRSYYASWYQQKPGQAPVLVIYGKNNRPSGIPDRFSGSGSGNTASLTITGAQAEDEADYYCAAWDDSLSGPVFGGGTKVTVLG

SEQ ID NO: 54 - protein, 243 aa, Artificial sequence
  Synthetic amino acid sequence
QVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 55 - DNA, 15 bp, Artificial sequence
  Synthetic DNA sequence
acctatactatgcac

SEQ ID NO: 56 - DNA, 51 bp, Artificial sequence
  Synthetic DNA sequence
gatatagcatatgatgggagtactaaatactacgcagactctgtgaagggc

SEQ ID NO: 57 - DNA, 33 bp, Artificial sequence
  Synthetic DNA sequence
gatgcagtggctggtgaagggtacttcgatctc

SEQ ID NO: 58 - DNA, 33 bp, Artificial sequence
  Synthetic DNA sequence
caaggagacagcctcagaagctattatgcaagt

SEQ ID NO: 59 - DNA, 21 bp, Artificial sequence
  Synthetic DNA sequence
aggaataatcagcggccctca

SEQ ID NO: 60 - DNA, 33 bp, Artificial sequence
  Synthetic DNA sequence
gcagcatgggatgacagcctgagtgcctgggtg

SEQ ID NO: 61 - DNA, 15 bp, Artificial sequence
  Synthetic DNA sequence
gcctctggcatgcac

SEQ ID NO: 62 - DNA, 51 bp, Artificial sequence
  Synthetic DNA sequence
tttatagcctatgatggaaatcaaaaattctatgcagactccgtgaagggc

SEQ ID NO: 63 - DNA, 30 bp, Artificial sequence
  Synthetic DNA sequence
gaaatgcaacgcgaggggtactttgactac

SEQ ID NO: 64 - DNA, 33 bp, Artificial sequence
  Synthetic DNA sequence
caaggagacagcctcagaagctattatgcaagc

SEQ ID NO: 65 - DNA, 21 bp, Artificial sequence
  Synthetic DNA sequence
ggtaaaaacaaccggccctca

SEQ ID NO: 66 - DNA, 33 bp, Artificial sequence
  Synthetic DNA sequence
gcaacatgggatgacaacctgagtggtccgata

SEQ ID NO: 67 - DNA, 15 bp, Artificial sequence
  Synthetic DNA sequence
gactactacatgagc

SEQ ID NO: 68 - DNA, 51 bp, Artificial sequence
  Synthetic DNA sequence
tacattagtactagtggtagtagcatatactatgtagactctgtgaagggc

SEQ ID NO: 69 - DNA, 30 bp, Artificial sequence
  Synthetic DNA sequence
gatcttcacggtgactatgcctttgactcc

SEQ ID NO: 70 - DNA, 33 bp, Artificial sequence
  Synthetic DNA sequence
caaggagacagtctcagaagttattatgcaagc

SEQ ID NO: 71 - DNA, 21 bp, Artificial sequence
  Synthetic DNA sequence
ggtaaaaacaaccggccctca

SEQ ID NO: 72 - DNA, 33 bp, Artificial sequence; identical to SEQ ID NO: 66
  Synthetic DNA sequence

SEQ ID NO: 73 - DNA, 15 bp, Artificial sequence
  Synthetic DNA sequence
agctatgccatgagc

SEQ ID NO: 74 - DNA, 51 bp, Artificial sequence
  Synthetic DNA sequence
gctattagtggtagtggtggtagcacatactacgcagactccgtgaagggc

SEQ ID NO: 75 - DNA, 30 bp, Artificial sequence
  Synthetic DNA sequence
gtgagcagcagctggtcccattttgactac

SEQ ID NO: 76 - DNA, 33 bp, Artificial sequence
  Synthetic DNA sequence
cgggccagtcagtatattagtaactggttggcc

SEQ ID NO: 77 - DNA, 21 bp, Artificial sequence
  Synthetic DNA sequence
aaggcgtctagtttagaaagt

SEQ ID NO: 78 - DNA, 30 bp, Artificial sequence
  Synthetic DNA sequence
caagagagttacaatacccccttattcact

SEQ ID NO: 79 - DNA, 15 bp, Artificial sequence
  Synthetic DNA sequence
aactatgccataaac

SEQ ID NO: 80 - DNA, 51 bp, Artificial sequence
  Synthetic DNA sequence
aacatacaccacgatggaaatggtaaatactatgtggactctgtggagggc

SEQ ID NO: 81 - DNA, 27 bp, Artificial sequence
  Synthetic DNA sequence
gacggctacgggggttaccttgacttg

SEQ ID NO: 82 - DNA, 33 bp, Artificial sequence; identical to SEQ ID NO: 64
  Synthetic DNA sequence

SEQ ID NO: 83 - DNA, 21 bp, Artificial sequence
  Synthetic DNA sequence
ggtaaaaacaaccggccctca

SEQ ID NO: 84 - DNA, 33 bp, Artificial sequence
  Synthetic DNA sequence
gcagcatgggatgacagcctgagtggtccggtg

SEQ ID NO: 85 - DNA, 15 bp, Artificial sequence
  Synthetic DNA sequence
agctatgctatgcac

SEQ ID NO: 86 - DNA, 51 bp, Artificial sequence
  Synthetic DNA sequence
gttatatcatatgatggaagcaataaatactacgcagactccgtgaagggc

SEQ ID NO: 87 - DNA, 33 bp, Artificial sequence
  Synthetic DNA sequence
gatctctcggggtacggtgactaccctgactac

SEQ ID NO: 88 - DNA, 33 bp, Artificial sequence; identical to SEQ ID NO: 64
  Synthetic DNA sequence

SEQ ID NO: 89 - DNA, 21 bp, Artificial sequence
  Synthetic DNA sequence
ggtagaaacgagcggccctca

SEQ ID NO: 90 - DNA, 33 bp, Artificial sequence
  Synthetic DNA sequence
gcagggtgggatgacagcctgactggtccggtg

SEQ ID NO: 91 - DNA, 357 bp, Artificial sequence
  Synthetic DNA sequence
caggtgcagctgcaggagtcggggggaggcttggtacagcctggggggtccctgagactctcctgtgcagcctctggattctccttcaacacctatactatgcactgggtccgccaggctccaggcaaggggctggagtgggtggcggatatagcatatgatgggagtactaaatactacgcagactctgtgaagggccgattcaccatctccagagacaatgccaagaactcactgtatctgcaaatgaacagcctgagagccgaggacacggctgtgtattactgtgcgagagatgcagtggctggtgaagggtacttcgatctctggggccgtggcaccctggtcaccgtctcc

SEQ ID NO: 92 - DNA, 333 bp, Artificial sequence
  Synthetic DNA sequence
tcgcagtctgctctgactcaggaccctgctgtgtctgtggccttgggacagacagtcaggatcacatgccaaggagacagcctcagaagctattatgcaagttggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtgcctgggtgttcggcggagggaccaagctgaccgtcctaggtgcg

SEQ ID NO: 93 - DNA, 354 bp, Artificial sequence
  Synthetic DNA sequence
caggtacagctgcagcagtcagggggaggcgtggtccagcctggggggtccctgagactctcctgtgcagcgtctgagttcaccttcagtgcctctggcatgcactgggtccgccaggctccaggcaagggcctggaatggatggcatttatagcctatgatggaaatcaaaaattctatgcagactccgtgaagggccgattcaccatctccagagacaattccaagaacactctgtatctgcaaatggacagcctgagaggtgaggacacggctgtgtattactgtgcgaaagaaatgcaacgcgaggggtactttgactactggggccagggaaccctggtcaccgtctcc

SEQ ID NO: 94 - DNA, 330 bp, Artificial sequence
  Synthetic DNA sequence
tcgaattttatgctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccaagtctggcaactcagcctccctggacatcagtgggctccagtctgaggatgaggctgattattattgtgcaacatgggatgacaacctgagtggtccgatattcggcggagggaccaaggtcaccgtcctaggt

SEQ ID NO: 95 - DNA, 354 bp, Artificial sequence
  Synthetic DNA sequence
caggtgcagctggcggagtctgggggaggcttggtcaagcctggagggtccctgagactctcctgtgcagcctctggattcaccttcagtgactactacatgagctggatccgccaggctccagggaaggggctggagtgggtttcatacattagtactagtggtagtagcatatactatgtagactctgtgaagggccgattcaccatctccagggacaacgccaagaactcactgtatctgcaaatggacagcctgagagacgacgacacggctgtttattactgtgcgagagatcttcacggtgactatgcctttgactcctggggccagggaaccctggtcaccgtctcc

SEQ ID NO: 96 - DNA, 327 bp, Artificial sequence
  Synthetic DNA sequence
tcgtctgagctgactcaggaccctgctgtgtctgtggccttgggacagacagtcaggatcacatgccaaggagacagtctcagaagttattatgcaagctggtaccagcagaagccaggacaggcccctgtacttgtcatctatggtaaaaacaaccggccctcagggatcccagaccgattctctggctccaagtctggcaactcagcctccctggacatcagtgggctccagtctgaggatgaggctgattattattgtgcaacatgggatgacaacctgagtggtccgatattcggcggagggaccaaggtcaccgtcctaggt

SEQ ID NO: 97 - DNA, 354 bp, Artificial sequence
  Synthetic DNA sequence
caggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtgagcagcagctggtcccattttgactactggggccagggaaccctggtcaccgtctcc

SEQ ID NO: 98 - DNA, 330 bp, Artificial sequence
  Synthetic DNA sequence
tcggatgttgtgatgactcagtctccttccaccctgtctgcatctgtaggagacagagtcaccatcacttgccgggccagtcagtatattagtaactggttggcctggtatcagcagaaaccagggaaagcccctaagctcctgatctataaggcgtctagtttagaaagtggggtcccatcaaggttcagcggcagtggatctgggacagagttcactctcaccatcagcagtctgcaacctgaagattttgcaacttactactgtcaagagagttacaatacccccttattcactttcggccctgggaccaagctggagatcaaacgt

SEQ ID NO: 99 - DNA, 351 bp, Artificial sequence
  Synthetic DNA sequence
caggtgcagctggtggagtctgggggaggcttagtggagcctggggggtccctgagactctcctgtgcggcctctggattcacctttagcaactatgccataaactgggtccgccaggctccagggaaggggctggagtgggtggccaacatacaccacgatggaaatggtaaatactatgtggactctgtggagggccgattcaccatctccagagacaacgccaagaattctctgtatctgcaaatggacagcctgagagccgaggacacggccatttattactgtgcgcgagacggctacgggggttaccttgacttgtggggccagggaaccctggtcaccgtctcc

SEQ ID NO: 100 - DNA, 327 bp, Artificial sequence
  Synthetic DNA sequence
tcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccggctcaggaaacacagcttccttgaccatcactggggctcaggcggaagatgaggctgactattactgtgcagcatgggatgacagcctgagtggtccggtgttcggcggagggaccaaggtcaccgtcctaggt

SEQ ID NO: 101 - DNA, 357 bp, Artificial sequence
  Synthetic DNA sequence
caggtgcagctgcaggagtcggggggaggcgtggtccagcctgggaggtccctgagactctcctgtgcagcctctcgattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatctctcggggtacggtgactaccctgactactggggccagggaaccctggtcaccgtctcc

SEQ ID NO: 102 - DNA, 327 bp, Artificial sequence
  Synthetic DNA sequence
tcgtctgagctgactcaggaccctgctgtgtctgtggccttgggacagacagtcagaatcacatgccaaggagacagcctcagaagctattatgcaagctggtaccagcagaagccaggacaggcccctgtacttgtcatgtatggtagaaacgagcggccctcaggggttcctgaccgattctctggctccaagtctggcacctctgcctccctggccatcagtggcctccagccagaggatgaggctaattattactgtgcagggtgggatgacagcctgactggtccggtgttcggcggagggaccaagctgaccgtcctaggt

SEQ ID NO: 103 - DNA, 735 bp, Artificial sequence
  Synthetic DNA sequence
caggtgcagctgcaggagtcggggggaggcttggtacagcctggggggtccctgagactctcctgtgcagcctctggattctccttcaacacctatactatgcactgggtccgccaggctccaggcaaggggctggagtgggtggcggatatagcatatgatgggagtactaaatactacgcagactctgtgaagggccgattcaccatctccagagacaatgccaagaactcactgtatctgcaaatgaacagcctgagagccgaggacacggctgtgtattactgtgcgagagatgcagtggctggtgaagggtacttcgatctctggggccgtggcaccctggtcaccgtctcctcaggtggaggcggttcaggcggaggtggctctggcggtggcggatcgcagtctgctctgactcaggaccctgctgtgtctgtggccttgggacagacagtcaggatcacatgccaaggagacagcctcagaagctattatgcaagttggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtgcctgggtgttcggcggagggaccaagctgaccgtcctaggtgcg

SEQ ID NO: 104 - DNA, 729 bp, Artificial sequence
  Synthetic DNA sequence
caggtacagctgcagcagtcagggggaggcgtggtccagcctggggggtccctgagactctcctgtgcagcgtctgagttcaccttcagtgcctctggcatgcactgggtccgccaggctccaggcaagggcctggaatggatggcatttatagcctatgatggaaatcaaaaattctatgcagactccgtgaagggccgattcaccatctccagagacaattccaagaacactctgtatctgcaaatggacagcctgagaggtgaggacacggctgtgtattactgtgcgaaagaaatgcaacgcgaggggtactttgactactggggccagggaaccctggtcaccgtctcctcaggtggaggcggttcaggcggaggtggctctggcggtggcggatcgaattttatgctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccaagtctggcaactcagcctccctggacatcagtgggctccagtctgaggatgaggctgattattattgtgcaacatgggatgacaacctgagtggtccgatattcggcggagggaccaaggtcaccgtcctaggt

SEQ ID NO: 105 - DNA, 726 bp, Artificial sequence
  Synthetic DNA sequence
caggtgcagctggcggagtctgggggaggcttggtcaagcctggagggtccctgagactctcctgtgcagcctctggattcaccttcagtgactactacatgagctggatccgccaggctccagggaaggggctggagtgggtttcatacattagtactagtggtagtagcatatactatgtagactctgtgaagggccgattcaccatctccagggacaacgccaagaactcactgtatctgcaaatggacagcctgagagacgacgacacggctgtttattactgtgcgagagatcttcacggtgactatgcctttgactcctggggccagggaaccctggtcaccgtctcctcaggtggaggcggttcaggcggaggtggctctggcggtggcggatcgtctgagctgactcaggaccctgctgtgtctgtggccttgggacagacagtcaggatcacatgccaaggagacagtctcagaagttattatgcaagctggtaccagcagaagccaggacaggcccctgtacttgtcatctatggtaaaaacaaccggccctcagggatcccagaccgattctctggctccaagtctggcaactcagcctccctggacatcagtgggctccagtctgaggatgaggctgattattattgtgcaacatgggatgacaacctgagtggtccgatattcggcggagggaccaaggtcaccgtcctaggt

SEQ ID NO: 106 - DNA, 729 bp, Artificial sequence
  Synthetic DNA sequence
caggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtgagcagcagctggtcccattttgactactggggccagggaaccctggtcaccgtctcctcaggtggaggcggttcaggcggaggtggctccggcggtggcggatcggatgttgtgatgactcagtctccttccaccctgtctgcatctgtaggagacagagtcaccatcacttgccgggccagtcagtatattagtaactggttggcctggtatcagcagaaaccagggaaagcccctaagctcctgatctataaggcgtctagtttagaaagtggggtcccatcaaggttcagcggcagtggatctgggacagagttcactctcaccatcagcagtctgcaacctgaagattttgcaacttactactgtcaagagagttacaatacccccttattcactttcggccctgggaccaagctggagatcaaacgt

SEQ ID NO: 107 - DNA, 723 bp, Artificial sequence
  Synthetic DNA sequence
caggtgcagctggtggagtctgggggaggcttagtggagcctggggggtccctgagactctcctgtgcggcctctggattcacctttagcaactatgccataaactgggtccgccaggctccagggaaggggctggagtgggtggccaacatacaccacgatggaaatggtaaatactatgtggactctgtggagggccgattcaccatctccagagacaacgccaagaattctctgtatctgcaaatggacagcctgagagccgaggacacggccatttattactgtgcgcgagacggctacgggggttaccttgacttgtggggccagggaaccctggtcaccgtctcctcaggtggaggcggttcaggcggaggtggctctggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccggctcaggaaacacagcttccttgaccatcactggggctcaggcggaagatgaggctgactattactgtgcagcatgggatgacagcctgagtggtccggtgttcggcggagggaccaaggtcaccgtcctaggt

SEQ ID NO: 108 - DNA, 729 bp, Artificial sequence
  Synthetic DNA sequence
caggtgcagctgcaggagtcggggggaggcgtggtccagcctgggaggtccctgagactctcctgtgcagcctctcgattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatctctcggggtacggtgactaccctgactactggggccagggaaccctggtcaccgtctcctcaggtggaggcggttcaggcggaggtggctctggcggtggcggatcgtctgagctgactcaggaccctgctgtgtctgtggccttgggacagacagtcagaatcacatgccaaggagacagcctcagaagctattatgcaagctggtaccagcagaagccaggacaggcccctgtacttgtcatgtatggtagaaacgagcggccctcaggggttcctgaccgattctctggctccaagtctggcacctctgcctccctggccatcagtggcctccagccagaggatgaggctaattattactgtgcagggtgggatgacagcctgactggtccggtgttcggcggagggaccaagctgaccgtcctaggt

SEQ ID NO: 109 - protein, 15 aa, Artificial sequence
  Synthetic amino acid sequence
SGGGGSGGGGSGGGG

SEQ ID NO: 110 - DNA, 45 bp, Artificial sequence
  Synthetic DNA sequence
tcaggtggaggcggttcaggcggaggtggctctggcggtggcgga

(19) United States
(12) Patent Application Publication
(10) Pub. No.: US 2013/0045206 A1
(43) Pub. Date: Feb. 21, 2013

(54) ANTIBODIES DIRECTED AGAINST THE TRANSFERRIN RECEPTOR AND USES THEREOF FOR IMMUNOTHERAPY OF IRON-DEPENDENT TUMOURS

(71) Applicants: Marie-Alix Poul, Montpellier (FR); Ronan Philippe Crepin, Buc (FR); James D. Marks, Kensington, CA (US)

(72) Inventors: Marie-Alix Poul, Montpellier (FR); Ronan Philippe Crepin, Buc (FR); James D. Marks, Kensington, CA (US)

(21) Appl. No.: 13/515,551

(22) PCT Filed: Dec. 16, 2010
(86) PCT No.: PCT/IB2010/055882

§ 371 Date: Nov. 6, 2012

(30) Foreign Application Priority Data
Dec. 16, 2009 (FR) 09/06090

(51) Int. Cl. C07K 16/28; C12N 15/63; A61P 37/06; C12N 5/10; A61K 39/395; A61P 35/00; C12N 15/13; C12N 1/21

(52) U.S. Cl. 424/135.1; 536/23.53; 435/320.1; 435/252.33; 435/331; 530/387.3

(57) ABSTRACT

The present invention relates to a molecular structure characterised in that said structure includes at least one amino acid sequence selected from: SEQ. ID NO.: 1, SEQ. ID NO.: 2, SEQ. ID NO.: 3, SEQ. ID NO.: 4, SEQ. ID NO.: 5, SEQ. ID NO.: 6, SEQ. ID NO.: 7, SEQ. ID NO.: 8, SEQ. ID NO.: 9, SEQ. ID NO.: 10, SEQ. ID NO.: 11, SEQ. ID NO.: 12, SEQ. ID NO.: 13, SEQ. ID NO.: 14, SEQ. ID NO.: 15, SEQ. ID NO.: 16, SEQ. ID NO.: 17, SEQ. ID NO.: 18, SEQ. ID NO.: 19, SEQ. ID NO.: 20, SEQ. ID NO.: 21, SEQ. ID NO.: 22, SEQ. ID NO.: 23, SEQ. ID NO.: 24, SEQ. ID NO.: 25, SEQ. ID NO.: 26, SEQ. ID NO.: 27, SEQ. ID NO.: 28, SEQ. ID NO.: 29, SEQ. ID NO.: 30, SEQ. ID NO.: 31, SEQ. ID NO.: 32, SEQ. ID NO.: 33, SEQ. ID NO.: 34, SEQ. ID NO.: 35, SEQ. ID NO.: 36, said amino acid sequence corresponding to an antigen complementarity determining region (CDR) of the variable domain of the heavy chain (CDR-H) or the light chain (CDR-L) of an antibody targeting the human transferrin receptor (TfR). The present invention also relates to a pharmaceutical composition including a therapeutically effective amount of at least one molecular structure as defined in the present application, combined with a pharmaceutically acceptable carrier.

The present invention relates to a novel family of completely human and cytotoxic antibodies directed against the transferrin receptor (anti-transferrin receptor antibodies) which induce the death of human haematopoietic cancer cells.

Transferrin (Tf) is a serum protein of 80 kDa, the role of which is to fix soluble iron. It is endocyted in cells due to its binding with the transferrin receptor (TfR). Acidification of the endosome causes a conformational change which salts out the iron in the cytosol. The Tf/TfR complex is then re-exported to the membrane, where the return to a physiological pH causes a dissociation of the Tf/TfR complex.

The human transferrin receptor (TfR), a cell proliferation marker, plays a predominant role in the absorption of iron in cells and in the regulation of cell growth. It has long been regarded as an interesting target for various pathologies, including cancer (since it is overexpressed via numerous highly proliferating cells) and cerebral diseases (since it is expressed via the haematoencephalic barrier), for therapeutic or diagnostic approaches.

Thus, in the course of the last 25 years, a certain number of murine anti-transferrin receptor (anti-TfR) monoclonal antibodies (mAb) have been developed by means of hybridoma technology, and have been tested for their inhibitory effect in vitro and sometimes in vivo on murine cancer models (Daniels et al., The transferrin receptor part I or part II, Clin. Immunol., 2006) or as carriers for brain targeting (Pardridge, Drug targeting to the brain, Pharm res 24: 1733, 2007).

In theory, the use of cytotoxic anti-transferrin receptor monoclonal antibodies (anti-TfR mAb) for immunotherapy of cancer would have to be limited by the undesirable effects induced on iron-dependent cells, such as highly proliferating haematopoietic lines.

However, a phase I trial carried out with mAb 42/6, which induces iron depletion, has proved to be encouraging, since no secondary effect was observed on patients during administration of the antibody (Brooks et al., Clin Cancer Res 1: 1259, 1995).

However, no response to cancer has been observed in patients with the 42/6 antibody. It is probable that the failure of this clinical trial was due to the foreign status (murine) of the human antimurine antibody (HAMA) which initiated the immune response in the patients, and to the rapid degradation of the antibody which takes place after repeated injections.

The murine antibody A24, which is an anti-TfR growth inhibitor mAb, is active on samples of acute and chronic forms of T cell leukaemia/lymphoma of adults in vitro (Moura et al., Blood 103: 1838, 2004; Callens et al., Leukemia 22: 42, 2008) and also on the development of mantle cell lymphoma in the mouse (Lepelletier et al., Cancer Res 67: 1145, 2007). mAb A24 interferes with the natural ligand of the TfR, charged diferric transferrin or holotransferrin (holo-Tf). A24 reduces endocytosis of holo-Tf and consequently the entry of iron into cells. Furthermore, mAb A24 reduces expression of TfR by disturbing the recycling of TfR to the surface of cells.

Among the anti-TfR mAb monoclonal antibodies described as cytotoxic in the literature, none of them inhibit the binding of transferrin to the TfR. Their antiproliferative or cytotoxic action results either from a bridging between several receptors inducing degradation of the TfR or from interaction of ADCC (antibody-dependent cell-mediated cytotoxicity) and CDC (complement-dependent cytotoxicity) (Daniels et al., The transferrin receptor part I or part II, Clin Immunol., 2006). Given that the TfR is expressed on the surface of certain quiescent cells (cells of the erythroid line, cerebrovascular endothelium cells, hepatocytes, renal tubule cells), the mode of action of these antibodies may present a certain cytotoxicity towards these cell types.

All the anti-TfR antibodies which exist to date are antibodies produced by immunization of animals. As therapeutic agents, they therefore have a potential not insignificant immunogenicity which limits their use.

At the present time there is no anti-TfR monoclonal antibody on the market. However, preclinical studies are in progress for the development of an anti-TfR chimaeric antibody, developed by MAT Biopharma and applied to metastatic melanoma. However, the anti-tumour activity of this antibody manifests itself by enlisting ADCC (antibody-dependent cell-mediated cytotoxicity) and CDC (complement-dependent cytotoxicity).

The treatments used to date in malignant haemopathies thus rest in part on the use of chemotherapies and pharmacological inhibitors directed against the tyrosine kinase activity of certain receptors. The chemotherapies are adapted as a function of the pathologies and the stage and grade of the tumours. The secondary effects are significant and are associated with the lack of specificity of the treatment. Pharmacological inhibitors prove to be more specific, but all the same have a toxicity caused by their binding to several families of receptors or intracellular signalling molecules of enzymatic activity. It is furthermore found that in certain patients resistances appear in the course of treatments associated with the acquisition of mutations.

Publication D1, “J. Mol. Biol. (2000), 301, 1149-1161”, deals with antibodies of human monomeric scFv format originating from a bank of phages which are capable of being internalized in SKBR-3 mammary tumour cells and not in normal cells of the same cell specificity. The antibody H7 described in D1, that is to say more particularly the fragment scFv H7 (scFv: “single chain fragment variable format”) is capable of inhibiting the binding of transferrin to the transferrin receptor, as well as the growth of SKBR-3 mammary tumour cells. The fragment scFv H7 reacts competitively with holotransferrin for binding with SKBR-3 cancer cells.

Publication D2, “Molecular Immunology 44 (2007), 3377-3788”, cites the six antibodies given as specific examples in the present application, obtaining of these in vitro by phage display in an scFv format and their selection for their capacity for being internalized specifically inside SKBR-3 mammary tumour cells.

These six antibodies are described for their specific binding with the transferrin receptor (with reference to the results of D1 mentioned above) and an efficacy of the six antibodies in the inhibition of the cell proliferation of SKBR-3 mammary tumour cells is assumed.

However, this assumption will be refuted in a publication D3, “Cancer Research, vol. 70, no. 13, July 2010”, where it is stated finally that the said antibodies do not inhibit the proliferation of these cells (page 5500, last paragraph of the left-hand column).

The determination of antibodies is thus only a first stage in the search for solutions to the treatment of pathologies such as cancer. In fact, it is then necessary to be able to determine on which tumour cell lines, and therefore for which therapeutic applications, the antibodies obtained are going to be able to be active, which often proves to be difficult given on the one hand the large number of cell lines and on the other hand the difficulty of predicting their behaviour.

The inventors have now found, surprisingly, that the antibodies according to the invention have a high potential for treatment of cancers of the lymphoma or leukaemia type and also as immunosuppressants for autoimmune diseases or the prevention of graft rejection.

One of the objects of the invention is therefore to provide novel therapeutic antibodies which target the human transferrin receptor (TfR) and which have a high potential for treatment of cancer, in particular of the lymphoma or leukaemia type.

Another object of the invention is to provide novel therapeutic antibodies which target the human transferrin receptor and which could be used for the treatment of autoimmune diseases or the prevention of graft rejection.

Another object of the invention is to provide novel antibodies which target the human transferrin receptor and which could be used for the vectorization of biologically active molecules, and more particularly of cytotoxic agent, within tumour cells which overexpress the TfR.

Another object of the invention is to provide novel antibodies which target the human transferrin receptor and which could be used for the vectorization of diagnostic molecules, and more particularly for cerebral imaging.

The novel antibodies according to the present invention prove to be molecules having a high therapeutic potential for the reasons given below and achieve all of the abovementioned objects.

In fact, the inventors of the present invention have developed novel completely human and cytotoxic monoclonal antibodies which are specific for the transferrin receptor (TfR), which inhibit the binding of transferrin (Tf) to the transferrin receptor (TfR) and which therefore deprive cells of iron. More particularly, the antibodies of the invention induce a depletion of iron in cells (drop in the intracellular concentration of iron), which results in an increase in iron on the receptor (TfR) at the surface of the cell.

The human antibodies according to the present invention particularly advantageously have an antiproliferative and cytotoxic activity towards cancer cells of haematopoietic origin in vitro as well as in vivo.

The present invention more particularly relates to a novel molecular construction, characterized in that it comprises at least one amino acid sequence chosen from:

SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36, the said amino acid sequence corresponding to a region determining complementarity with the antigen (“CDR”: “complementarity determining region”) of the variable domain of the heavy chain (CDR-H) or of the light chain (CDR-L) of an antibody which targets the human transferrin receptor (TfR).

Molecular construction is understood as meaning any construction (such as, for example, a molecule, an antibody or an antibody fragment or other) which could be prepared by the person skilled in the art and which comprises any one of the amino acid sequences SEQ ID NO: 1 to 36 as defined in the present application.

The molecular construction of the invention is more particularly made up of at least one of the amino acid sequences chosen from: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 and SEQ ID NO: 36.

The sequences thus defined more particularly correspond to the CDR located in the position numbered 3 in each heavy chain (CDR3-H) or in each light chain (CDR3-L) of an antibody which targets the human transferrin receptor (TfR).

More particularly, the sequences SEQ ID NO: 3, SEQ ID NO: 9, SEQ ID NO: 15, SEQ ID NO: 21, SEQ ID NO: 27 and SEQ ID NO: 33 correspond to “CDR3-H” and the sequences SEQ ID NO: 6, SEQ ID NO: 12, SEQ ID NO: 18, SEQ ID NO: 24, SEQ ID NO: 30 and SEQ ID NO: 36 correspond to “CDR3-L”.

According to the invention, at least one of the sequences SEQ ID NO: 1 to SEQ ID NO: 36 of the molecular construction is included in one of the amino acid sequences chosen from:

### SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 and SEQ ID NO: 48,

each of the said sequences SEQ ID NO: 37 to SEQ ID NO: 48 corresponding to the variable domain of the heavy chain (VH) or of the light chain (VL) of an antibody which targets the human transferrin receptor (TfR), the said sequence SEQ ID NO: 37 comprising the sequences SEQ ID NO: 1, SEQ ID NO: 2 and SEQ ID NO: 3, the said sequence SEQ ID NO: 38 comprising the sequences SEQ ID NO: 4, SEQ ID NO: 5 and SEQ ID NO: 6, the said sequence SEQ ID NO: 39 comprising the sequences SEQ ID NO: 7, SEQ ID NO: 8 and SEQ ID NO: 9, the said sequence SEQ ID NO: 40 comprising the sequences SEQ ID NO: 10, SEQ ID NO: 11 and SEQ ID NO: 12, the said sequence SEQ ID NO: 41 comprising the sequences SEQ ID NO: 13, SEQ ID NO: 14 and SEQ ID NO: 15, the said sequence SEQ ID NO: 42 comprising the sequences SEQ ID NO: 16, SEQ ID NO: 17 and SEQ ID NO: 18, the said sequence SEQ ID NO: 43 comprising the sequences SEQ ID NO: 19, SEQ ID NO: 20 and SEQ ID NO: 21, the said sequence SEQ ID NO: 44 comprising the sequences SEQ ID NO: 22, SEQ ID NO: 23 and SEQ ID NO: 24, the said sequence SEQ ID NO: 45 comprising the sequences SEQ ID NO: 25, SEQ ID NO: 26 and SEQ ID NO: 27, the said sequence SEQ ID NO: 46 comprising the sequences SEQ ID NO: 28, SEQ ID NO: 29 and SEQ ID NO: 30, the said sequence SEQ ID NO: 47 comprising the sequences SEQ ID NO: 31, SEQ ID NO: 32 and SEQ ID NO: 33, the said sequence SEQ ID NO: 48 comprising the sequences SEQ ID NO: 34, SEQ ID NO: 35 and SEQ ID NO: 36.

Furthermore, according to the invention at least one of the sequences SEQ ID NO: 37 to SEQ ID NO: 48 is included in one of the amino acid sequences chosen from:

### SEQ ID NO: 49, SEQ ID NO: 50, SEQ ID NO: 51, SEQ ID NO: 52, SEQ ID NO: 53 and SEQ ID NO: 54,

each of the said sequences SEQ ID NO: 49 to SEQ ID NO: 54 comprising the variable domain of the heavy chain (VH) and of the light chain (VL) of an antibody which targets the human transferrin receptor (TfR),
the said sequence SEQ ID NO: 49 comprising the sequences SEQ ID NO: 37 and SEQ ID NO: 38 linked to one another by the peptide linker having an amino acid sequence SEQ ID NO: 109,
the said sequence SEQ ID NO: 50 comprising the sequences SEQ ID NO: 39 and SEQ ID NO: 40 linked to one another by the peptide linker SEQ ID NO: 109,
the said sequence SEQ ID NO: 51 comprising the sequences SEQ ID NO: 41 and SEQ ID NO: 42 linked to one another by the peptide linker SEQ ID NO: 109,
the said sequence SEQ ID NO: 52 comprising the sequences SEQ ID NO: 43 and SEQ ID NO: 44 linked to one another by the peptide linker SEQ ID NO: 109,
the said sequence SEQ ID NO: 53 comprising the sequences SEQ ID NO: 45 and SEQ ID NO: 46 linked to one another by the peptide linker SEQ ID NO: 109,
the said sequence SEQ ID NO: 54 comprising the sequences SEQ ID NO: 47 and SEQ ID NO: 48 linked to one another by the peptide linker SEQ ID NO: 109.

The various sequences SEQ ID NO: 37 to SEQ ID NO: 48 mentioned above could also be bound to one another by a peptide which has a different sequence to SEQ ID NO: 109.

If the size of the peptide linker is decreased, for example, molecules of the “diabodies” type would then be obtained. However, an increase in the size of the peptide linker may also be envisaged.

The sequences SEQ ID NO: 49 to SEQ ID NO: 54 each comprise the total variable domain of the heavy chain and of the light chain of an antibody which targets the human transferrin receptor (TfR).

The sequences SEQ ID NO: 37, SEQ ID NO: 39, SEQ ID NO: 41, SEQ ID NO: 43, SEQ ID NO: 45 and SEQ ID NO: 47 each define the total variable domain of the heavy chain (VH) of an antibody which targets the human transferrin receptor (TfR).

The sequences SEQ ID NO: 38, SEQ ID NO: 40, SEQ ID NO: 42, SEQ ID NO: 44, SEQ ID NO: 46 and SEQ ID NO: 48 each define the total variable domain of the light chain (VL) of an antibody which targets the human transferrin receptor (TfR).

The SEQ ID NO: 1 to 3, 7 to 9, 13 to 15, 19 to 21, 25 to 27 and 31 to 33 each define a region determining the complementarity with the antigen of the variable domain of the heavy chain (CDR-H) of an antibody which targets the human transferrin receptor (TfR).

The SEQ ID NO: 4 to 6, 10 to 12, 16 to 18, 22 to 24, 28 to 30 and 34 to 36 each define a region determining the complementarity with the antigen of the variable domain of the light chain (CDR-L) of an antibody which targets the human transferrin receptor (TfR).

The sequences SEQ ID NO: 1 to SEQ ID NO: 54 thus relates to the total or only a part of the variable domain of the heavy and/or light chain of an antibody which targets the human transferrin receptor (TfR).

According to an advantageous embodiment of the invention, the molecular construction as defined above is in the form of a monomer.

According to another advantageous embodiment of the invention, the molecular construction as defined above is in the form of a dimer.

According to still another advantageous embodiment of the invention, the molecular construction as defined above also comprises a fragment Fc.

Preferably, according to the invention, the molecular construction as defined above is more particularly an antibody or an antibody fragment.

The sequences SEQ ID NO: 49 to SEQ ID NO: 54 as defined in the present application more precisely define antibody fragments and not “complete” antibodies, since these sequences do not comprise the constant domain found in the natural antibodies (immunoglobins).

Generally, one antibody differs from another in the primary sequence of its variable domain of the heavy chain (VH) and of the light chain (VL). The sequence of the constant domain is identical for a given class or sub-class of antibody.

Thus, to be entirely precise, the term “antibody” as used above and below to define the subject matter of the present invention should more particularly be understood as meaning “antibody fragment”.

The antibodies or antibody fragments of the invention are all specific for TfR but do not have the same primary sequences (see SEQ ID NO: 49 to SEQ ID NO: 54). The said antibodies differ in their paratope, that is to say the contact zone of the epitope on the antigen, in this case TfR.

The present invention also relates to nucleotide sequences, characterized in that they code respectively for the amino acid sequences SEQ ID NO: 1 to SEQ ID NO: 36 as defined above, and in that they are represented respectively by the sequences SEQ ID NO: 55 to SEQ ID NO: 90.

SEQ ID NO: 55 thus represents the nucleotide sequence coding for the amino acid sequence SEQ ID NO: 1 and so on (SEQ ID NO: 56 coding for SEQ ID NO: 2, SEQ ID NO: 57 coding for SEQ ID NO: 3, SEQ ID NO: 58 coding for SEQ ID NO: 4, SEQ ID NO: 59 coding for SEQ ID NO: 5, SEQ ID NO: 60 coding for SEQ ID NO: 6, SEQ ID NO: 61 coding for SEQ ID NO: 7, SEQ ID NO: 62 coding for SEQ ID NO: 8, SEQ ID NO: 63 coding for SEQ ID NO: 9, SEQ ID NO: 64 coding for SEQ ID NO: 10, SEQ ID NO: 65 coding for SEQ ID NO: 11, SEQ ID NO: 66 coding for SEQ ID NO: 12, SEQ ID NO: 67 coding for SEQ ID NO: 13, SEQ ID NO: 68 coding for SEQ ID NO: 14, SEQ ID NO: 69 coding for SEQ ID NO: 15, SEQ ID NO: 70 coding for SEQ ID NO: 16, SEQ ID NO: 71 coding for SEQ ID NO: 17, SEQ ID NO: 72 coding for SEQ ID NO: 18, SEQ ID NO: 73 coding for SEQ ID NO: 19, SEQ ID NO: 74 coding for SEQ ID NO: 20, SEQ ID NO: 75 coding for SEQ ID NO: 21, SEQ ID NO: 76 coding for SEQ ID NO: 22, SEQ ID NO: 77 coding for SEQ ID NO: 23, SEQ ID NO: 78 coding for SEQ ID NO: 24, SEQ ID NO: 79 coding for SEQ ID NO: 25, SEQ ID NO: 80 coding for SEQ ID NO: 26, SEQ ID NO: 81 coding for SEQ ID NO: 27, SEQ ID NO: 82 coding for SEQ ID NO: 28, SEQ ID NO: 83 coding for SEQ ID NO: 29, SEQ ID NO: 30 coding for SEQ ID NO: 84, SEQ ID NO: 31 coding for SEQ ID NO: 85, SEQ ID NO: 32 coding for SEQ ID NO: 86, SEQ ID NO: 33 coding for SEQ ID NO: 87, SEQ ID NO: 34 coding for SEQ ID NO: 88, SEQ ID NO: 35 coding for SEQ ID NO: 89 and SEQ ID NO: 36 coding for SEQ ID NO: 90).

The present invention also relates to nucleotide sequences, characterized in that they code respectively for the amino acid sequences SEQ ID NO: 37 to SEQ ID NO: 48 as defined above, and in that they are represented respectively by the sequences SEQ ID NO: 91 to SEQ ID NO: 102.

SEQ ID NO: 91 thus represents the nucleotide sequence coding for the amino acid sequence SEQ ID NO: 37 and so on (SEQ ID NO: 92 coding for SEQ ID NO: 38, SEQ ID NO: 93 coding for SEQ ID NO: 39, SEQ ID NO: 94 coding for SEQ ID NO: 40, SEQ ID NO: 95 coding for SEQ ID NO: 41, SEQ ID NO: 96 coding for SEQ ID NO: 42, SEQ ID NO: 97 coding for SEQ ID NO: 43, SEQ ID NO: 98 coding for SEQ ID NO: 44, SEQ ID NO: 99 coding for SEQ ID NO: 45, SEQ ID NO: 100 coding SEQ ID NO: 46, SEQ ID NO: 101 coding for SEQ ID NO: 47, SEQ ID NO: 102 coding for SEQ ID NO: 48).

The invention also relates to nucleotide sequences, characterized in that they code respectively for the amino acid sequences SEQ ID NO: 49 to SEQ ID NO: 54 as defined above, and in that they are represented respectively by the sequences SEQ ID NO: 103 to SEQ ID NO: 108,

the said SEQ ID NO: 103 comprising the sequences SEQ ID NO: 91 and SEQ ID NO: 92 linked to one another by the peptide linker having a nucleotide sequence SEQ ID NO: 110, the said SEQ ID NO: 104 comprising the sequences SEQ ID NO: 93 and SEQ ID NO: 94 linked to one another by SEQ ID NO: 110,
the said SEQ ID NO: 105 comprising the sequences SEQ ID NO: 95 and SEQ ID NO: 96 linked to one another by SEQ ID NO: 110,
the said SEQ ID NO: 106 comprising the sequences SEQ ID NO: 97 and SEQ ID NO: 98 linked to one another by SEQ ID NO: 110,
the said SEQ ID NO: 107 comprising the sequences SEQ ID NO: 99 and SEQ ID NO: 100 linked to one another by SEQ ID NO: 110,
the said SEQ ID NO: 108 comprising the sequences SEQ ID NO: 10 and SEQ ID NO: 102 linked to one another by SEQ ID NO: 110,
SEQ ID NO: 103 representing the nucleotide sequence coding for the amino acid sequence SEQ ID NO: 49 and so on (SEQ ID NO: 104 coding for SEQ ID NO: 50, SEQ ID NO: 105 coding for SEQ ID NO: 51, SEQ ID NO: 106 coding for SEQ ID NO: 52, SEQ ID NO: 107 coding for SEQ ID NO: 53 and SEQ ID NO: 108 coding for SEQ ID NO: 54).

The invention also relates to an isolated nucleic acid molecule comprising at least one of the nucleotide sequences SEQ ID NO: 55 to SEQ ID NO: 108 as defined above.

The invention furthermore also relates to:
an expression vector comprising a nucleic acid molecule as defined above,
a host cell or an organism comprising an expression vector as defined above.

The invention also relates to a pharmaceutical composition comprising a therapeutically effective amount of at least one molecular construction as defined above in combination with a pharmaceutically acceptable carrier.

As described above, the molecular construction is preferably an antibody or an antibody fragment.

According to an advantageous embodiment, the molecular construction is used in the said pharmaceutical composition to vectorize one (or more) biologically active molecule(s).

The antibodies of the invention have internalizing properties (see Example 1), which make them an interesting tool for playing the role of vectors for delivery of the said biologically active molecules to the inside of cancer cells which overexpress the TfR.

According to another advantageous embodiment, the molecular construction is used in the said pharmaceutical composition for targeting liposomes (formation of “immunoliposomes”) or nanoparticles charged with one (or more) cytotoxic agent(s) and/or one (or more) agent(s) with a diagnostic aim.

The invention also relates to a pharmaceutical composition as defined above for its use as a medicament in the treatment of pathologies with an overexpression of the TfR.

An example which may be mentioned of pathologies with an overexpression of the TfR is cancer, in particular of the lymphoma or leukaemia type.

According to an advantageous embodiment of the invention, the treatment against cancer more particularly takes place:
by an antiproliferative and cytotoxic action of the antibodies of the invention towards cancer cells in vitro and/or in vivo,
   and/or
by the antibodies of the invention inducing the death of cancer cells (by depriving them of iron).

The present invention also relates to a method for inhibiting the cell proliferation of cancer cells and/or for inducing the death of the said cells, characterized in that it comprises administration to a patient of at least one substance chosen from:
a molecular construction as defined above,
a pharmaceutical composition as defined above,
   in order to bind the said substance to the said cancer cells and thus to cause inhibition of the proliferation and/or to induce the death of cancer cells.

The present invention also relates to a method for treatment of cancer, characterized in that it comprises administration to a patient of at least one substance chosen from:
a molecular construction as defined above,
a pharmaceutical composition as defined above,
   in order to bind the said substance to the transferrin receptor of cancer cells and thus to cause inhibition of the proliferation and/or to induce the death of cancer cells.

Examples of cancer cells which may be mentioned are cells of haematopoietic origin.

Another example of pathologies with an overexpression of the TfR is that of autoimmune pathologies.

In fact, in this case in point these are cell effectors with an overexpression of the TfR.

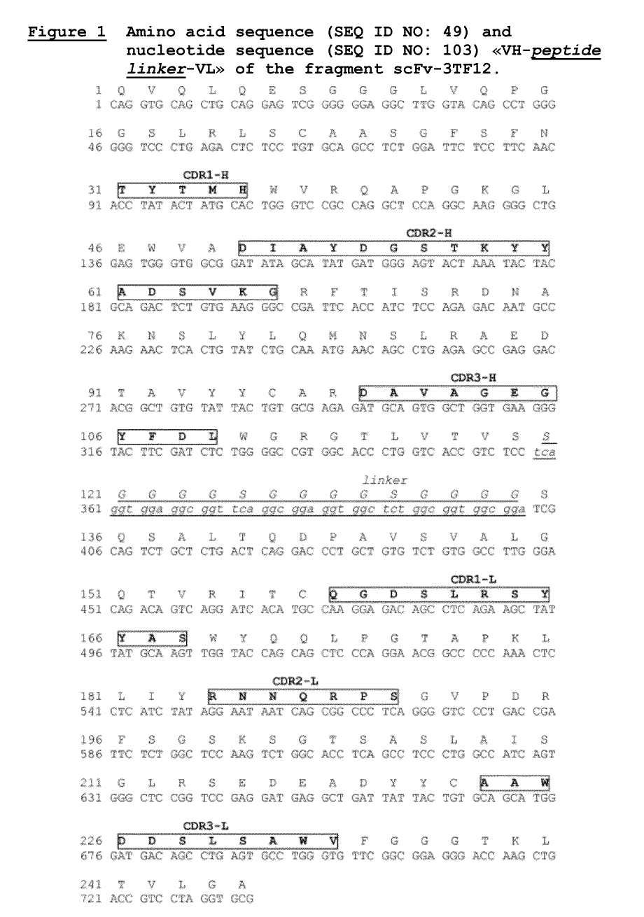

FIG. 1 shows the amino acid sequence (SEQ ID NO: 49) and the nucleotide sequence (SEQ ID NO: 103) of the variable domain of the heavy chain (VH) and of the light chain (VL) of the monovalent fragment of the invention called “scFv-3TF12”, the heavy and light chain being linked to one another by a peptide linker.

Each line above this represents SEQ ID NO: 49 and each line below this represents SEQ ID NO: 103.

The 3 “CDR” regions of the heavy chain (CDR1-H to CDR3-H: SEQ ID NO: 1 to SEQ ID NO: 3) and the 3 “CDR” regions of the light chain (CDR1-L to CDR3-L: SEQ ID NO: 4 to SEQ ID NO: 6) of the sequence SEQ ID NO: 49 are enclosed in a box and shown in bold.

The heavy chain (VH) comprises amino acid no. 1 to amino acid no. 119 (SEQ ID NO: 37).

The light chain (VL) comprises amino acid no. 135 to amino acid no. 245 (SEQ ID NO: 38).

The peptide linker (SEQ ID NO: 109) comprises 15 amino acids no. 120 to 134, which are shown in italics and underlined (S G G G G S G G G G S G G G G).

The nucleotide sequence SEQ ID NO: 110 which codes for SEQ ID NO: 109 is in italics, underlined and is in lower case.

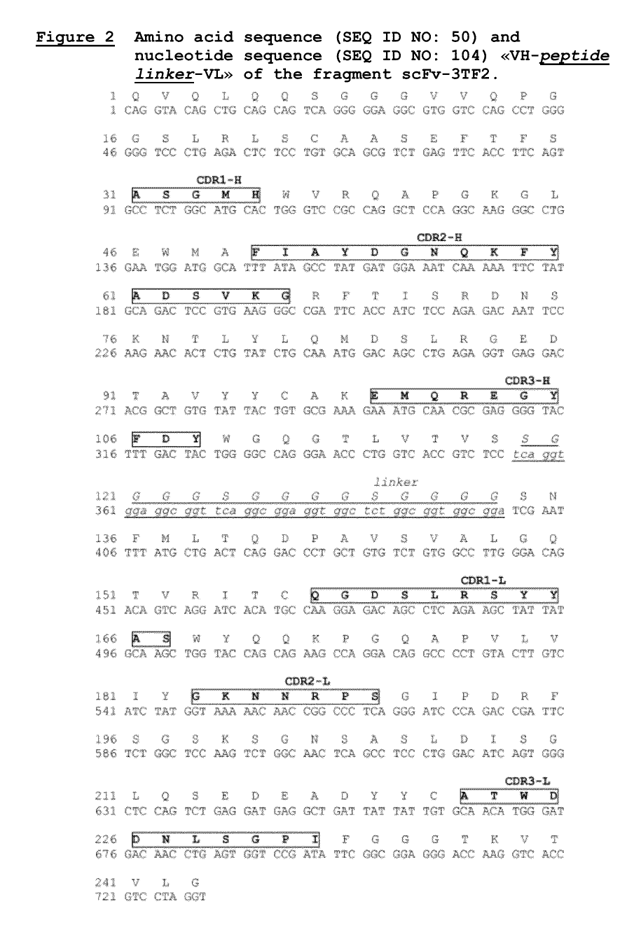

FIG. 2 shows the amino acid sequence (SEQ ID NO: 50) and the nucleotide sequence (SEQ ID NO: 104) of the variable domain of the heavy chain (VH) and of the light chain (VL) of the monovalent fragment of the invention called “scFv-3TF2”, the heavy and light chain being linked to one another by a peptide linker.

Each line above this represents SEQ ID NO: 50 and each line below this represents SEQ ID NO: 104.

The 3 “CDR” regions of the heavy chain (CDR1-H to CDR3-H: SEQ ID NO: 7 to SEQ ID NO: 9) and the 3 “CDR” regions of the light chain (CDR1-L to CDR3-L: SEQ ID NO: 10 to SEQ ID NO: 12) of the sequence SEQ ID NO: 50 are enclosed in a box and shown in bold.

The heavy chain (VH) comprises amino acid no. 1 to amino acid no. 118 (SEQ ID NO: 39).

The light chain (VL) comprises amino acid no. 134 to amino acid no. 243 (SEQ ID NO: 40).

The peptide linker (SEQ ID NO: 109) is as defined in FIG. 1: it comprises 15 amino acids ranging from amino acid no. 119 to 133.

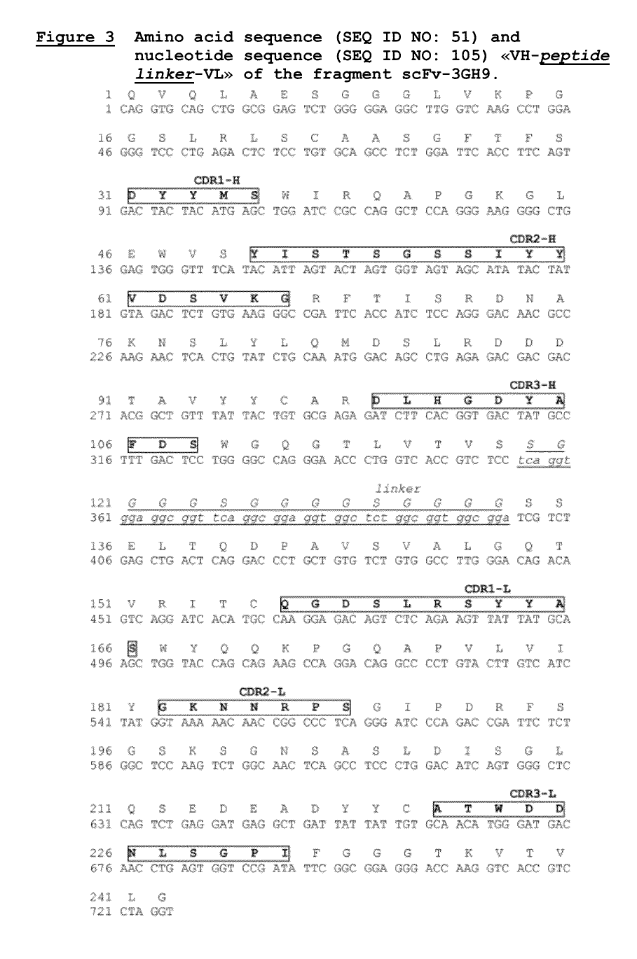

FIG. 3 shows the amino acid sequence (SEQ ID NO: 51) and the nucleotide sequence (SEQ ID NO: 105) of the variable domain of the heavy chain (VH) and of the light chain (VL) of the monovalent fragment of the invention called “scFv-3 GH9”, the heavy and light chain being linked to one another by a peptide linker.

Each line above this represents SEQ ID NO: 51 and each line below this represents SEQ ID NO: 105.

The 3 “CDR” regions of the heavy chain (CDR1-H to CDR3-H: SEQ ID NO: 13 to SEQ ID NO: 15) and the 3 “CDR” regions of the light chain (CDR1-L to CDR3-L: SEQ ID NO: 16 to SEQ ID NO: 18) of the sequence SEQ ID NO: 51 are enclosed in a box and shown in bold.

The heavy chain (VH) comprises amino acid no. 1 to amino acid no. 118 (SEQ ID NO: 41).

The light chain (VL) comprises amino acid no. 134 to amino acid no. 242 (SEQ ID NO: 42).

The peptide linker (SEQ ID NO: 109) is as defined in FIG. 1: it comprises fifteen amino acids ranging from amino acid no. 119 to 133.

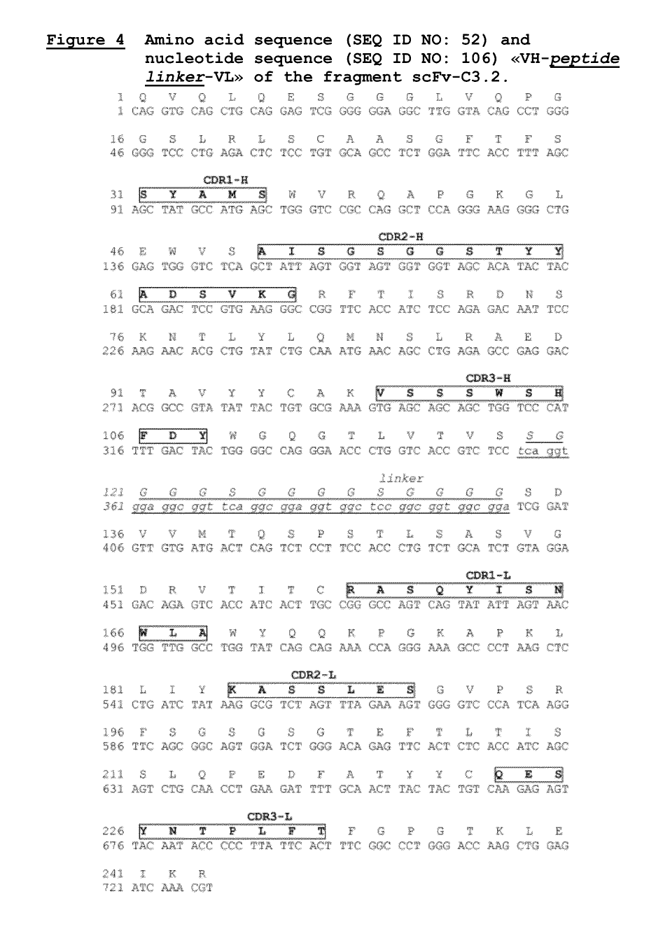

FIG. 4 shows the amino acid sequence (SEQ ID NO: 52) and the nucleotide sequence (SEQ ID NO: 106) of the variable domain of the heavy chain (VH) and of the light chain (VL) of the monovalent fragment of the invention called “scFv-C3.2”, the heavy and light chain being linked to one another by a peptide linker.

Each line above this represents SEQ ID NO: 52 and each line below this represents SEQ ID NO: 106.

The 3 “CDR” regions of the heavy chain (CDR1-H to CDR3-H: SEQ ID NO: 19 to SEQ ID NO: 21) and the 3 “CDR” regions of the light chain (CDR1-L to CDR3-L: SEQ ID NO: 22 to SEQ ID NO: 24) of the sequence SEQ ID NO: 52 are enclosed in a box and shown in bold.

The heavy chain (VH) comprises amino acid no. 1 to amino acid no. 118 (SEQ ID NO: 43).

The light chain (VL) comprises amino acid no. 134 to amino acid no. 243 (SEQ ID NO: 44).

The peptide linker (SEQ ID NO: 109) is as defined in FIG. 1: it comprises 15 amino acids ranging from amino acid no. 119 to 133.

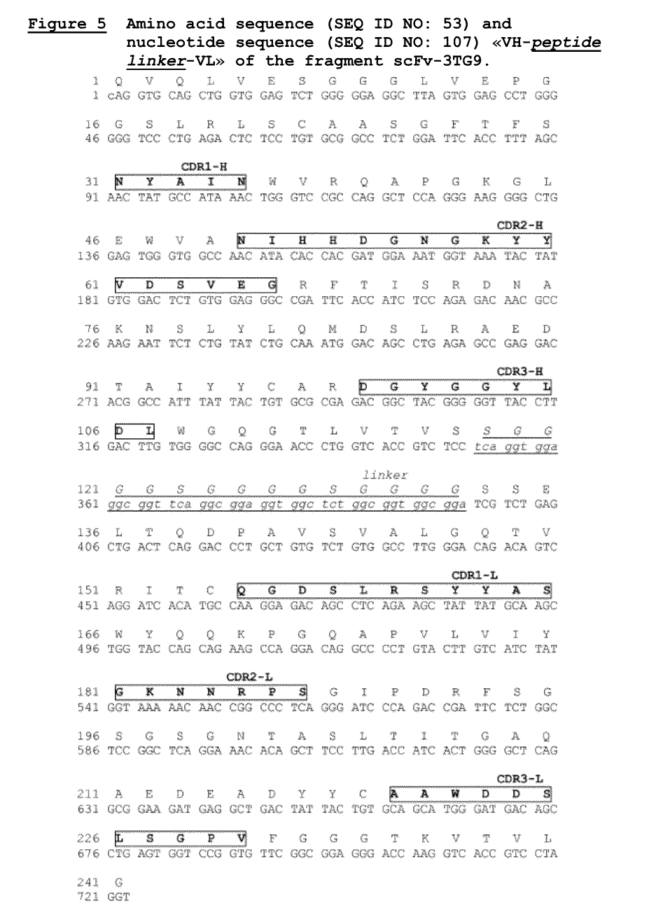

FIG. 5 shows the amino acid sequence (SEQ ID NO: 53) and the nucleotide sequence (SEQ ID NO: 107) of the variable domain of the heavy chain (VH) and of the light chain (VL) of the monovalent fragment of the invention called “scFv-3TG9”, the heavy and light chain being linked to one another by a peptide linker.

Each line above this represents SEQ ID NO: 53 and each line below this represents SEQ ID NO: 107.

The 3 “CDR” regions of the heavy chain (CDR1-H to CDR3-H: SEQ ID NO: 25 to SEQ ID NO: 27) and the 3 “CDR” regions of the light chain (CDR1-L to CDR3-L: SEQ ID NO: 28 to SEQ ID NO: 30) of the sequence SEQ ID NO: 53 are enclosed in a box and shown in bold.

The heavy chain (VH) comprises amino acid no. 1 to amino acid no. 117 (SEQ ID NO: 45).

The light chain (VL) comprises amino acid no. 133 to amino acid no. 241 (SEQ ID NO: 46).

The peptide linker (SEQ ID NO: 109) is as defined in FIG. 1: it comprises the amino acid no. 118 to 132.

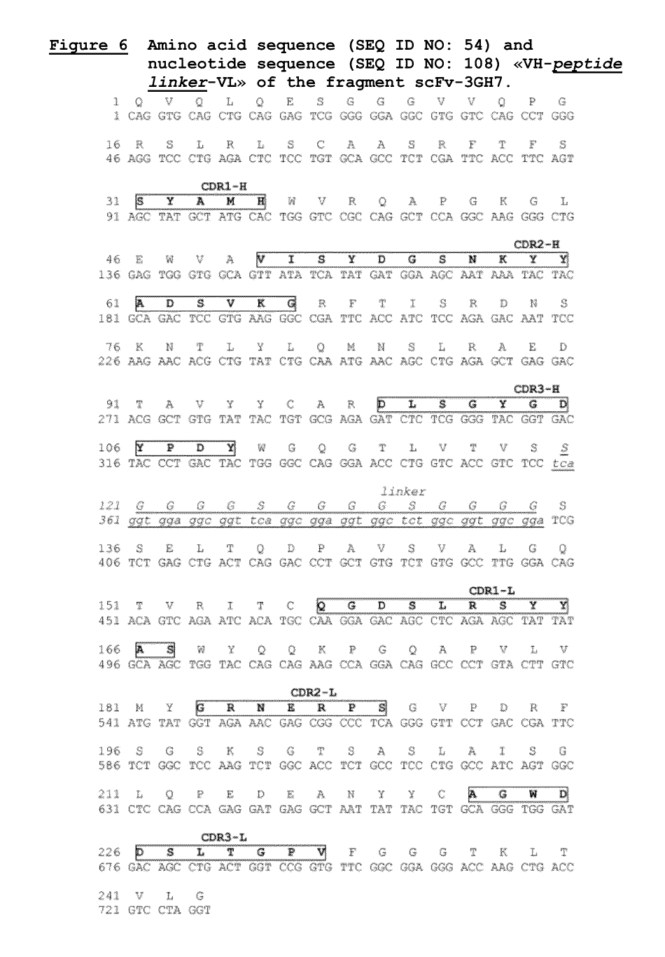

FIG. 6 shows the amino acid sequence (SEQ ID NO: 54) and the nucleotide sequence (SEQ ID NO: 108) of the variable domain of the heavy chain (VH) and of the light chain (VL) of the monovalent fragment of the invention called “scFv-3 GH7”, the heavy and light chain being linked to one another by a peptide linker.

Each line above this represents SEQ ID NO: 54 and each line below this represents SEQ ID NO: 108.

The 3 “CDR” regions of the heavy chain (CDR1-H to CDR3-H: SEQ ID NO: 31 to SEQ ID NO: 33) and the 3 “CDR” regions of the light chain (CDR1-L to CDR3-L: SEQ ID NO: 34 to SEQ ID NO: 36) of the sequence SEQ ID NO: 54 are enclosed in a box and shown in bold.

The heavy chain (VH) comprises amino acid no. 1 to amino acid no. 119 (SEQ ID NO: 47).

The light chain (VL) comprises amino acid no. 135 to amino acid no. 243 (SEQ ID NO: 48).

The peptide linker (SEQ ID NO: 109) is as defined in FIG. 1: it comprises 15 amino acids ranging from amino acid no. 120 to 134.

FIG. 7 illustrates the specificity of the six antibodies of the invention for the TfR of sequence SEQ ID NO: 49 to SEQ ID NO: 54 as defined above and described in the preceding FIGS. 1 to 6.

LS-174T cells were incubated for one hour at 4° C. with various concentrations of holotransferrin (Sigma) at the concentrations indicated.

The binding of the anti-TfR scFv phages or of the control phage scFV-F5 specific for the receptor ErBb2 present on the LS-174T cells is quantified with the aid of a murine antibody directed against the protein p8 of the phage capsid (anti-M13, GE Healthcare) and then measured by FACS with the aid of a fluorescent antibody which recognizes murine immunoglobulins.

The MFI (%) signal represents the mean fluorescence intensity (MFI) in relation to the MFI obtained in the absence of transferrin competitor.

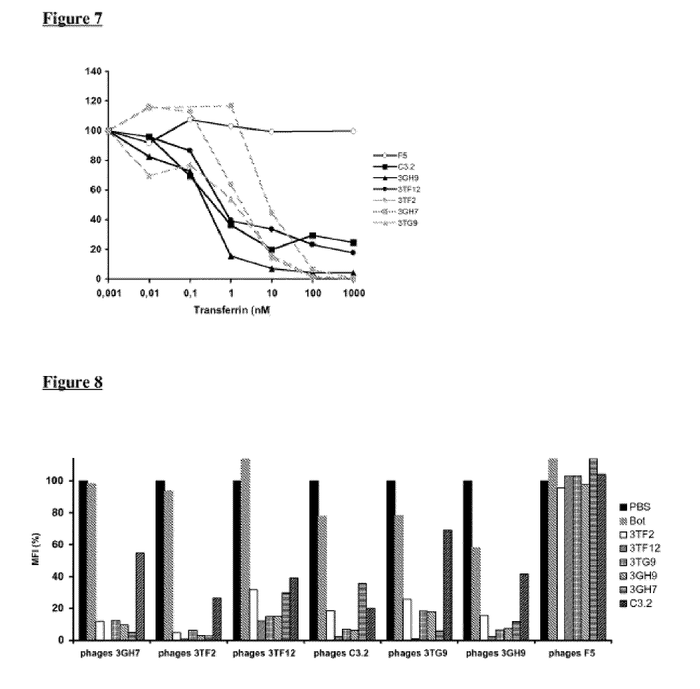

FIG. 8 shows the epitope map of the epitopes of the six anti-TfR antibodies of the invention (anti-TfR scFVs) of SEQ ID NO: 49 to SEQ ID NO: 54.

LS-174T tumour cells which overexpress the TfR were incubated in the presence of 10 μg/ml of soluble anti-TfR antibody or a control antibody directed against botulinum toxin (Bot) for one hour at 4° C. 10¹¹ cfu/ml of antibody phages (scFv phages) were then added to the cells for one hour at 4° C.

The binding of the anti-TfR scFv phages or the control phage scFv-F5 specific for the receptor ErBb2 is quantified in the same manner as described in FIG. 7.

The MFI (%) signal represents the mean fluorescence intensity (MFI) in relation to the MFI obtained in the absence of antibody competitor.

FIG. 9 illustrates the inhibition of the Tf/TfR binding by the six antibodies “anti-TfR scFvs” of the invention.

LS-174T cells were incubated in the presence of anti-TfR scFv antibodies at the concentration of 20 μg/ml for one hour at 4° C. Alternatively, an scFv antibody which is not specific for TfR is used (Bot), or also incubation in PBS alone. Five hundred nM fluorescent holotransferrin (Tf-FITC) was then added for one hour at 4° C. After washing, the Tf-FITC bound to the cells was measured by FACS. The signal has been shown in the MFI percentage in relation to the MFI of cells incubated without antibody (PBS)

FIG. 10 illustrates the proliferation test on the Jurkat line (produced by a human T lymphoma) for 3 days in the presence of the six antibodies “anti-TfR scFvs” of the invention.

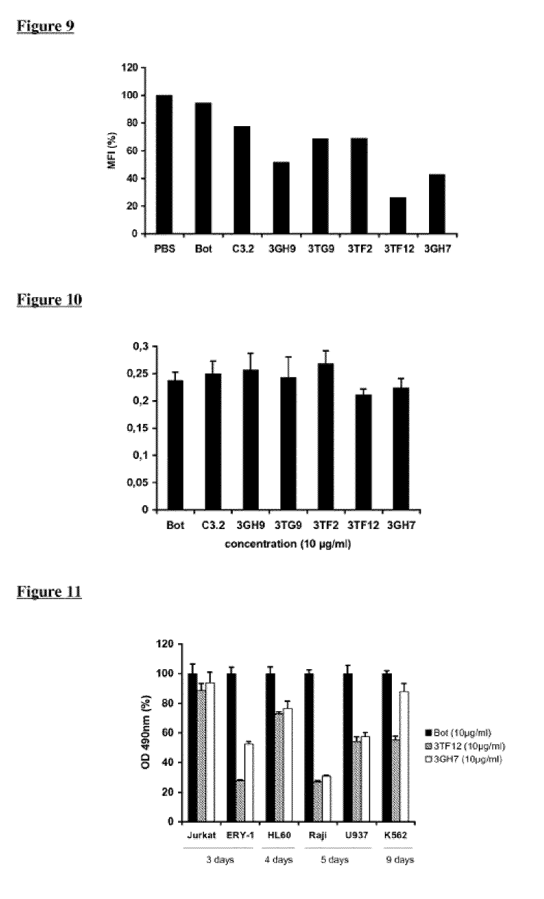

FIG. 11 illustrates the viability tests on several haematopoietic cancer lines in the presence of anti-TfR scFvs, that is to say 3TF12 and 3 GH7.

FIGS. 10 and 11: A 96-well plate was seeded with 5,000 Jurkat cells per well in sixplicate in RPMI/SVF 10% medium containing 10 μg/ml of soluble antibodies. The cells were incubated in a humidity chamber with 5% CO₂ at 37° C. for 3 days. The number of viable cells was quantified with the aid of an MTT test (CellTiter 96 Aqueous One Solution Cell Proliferation Assay, Promega).

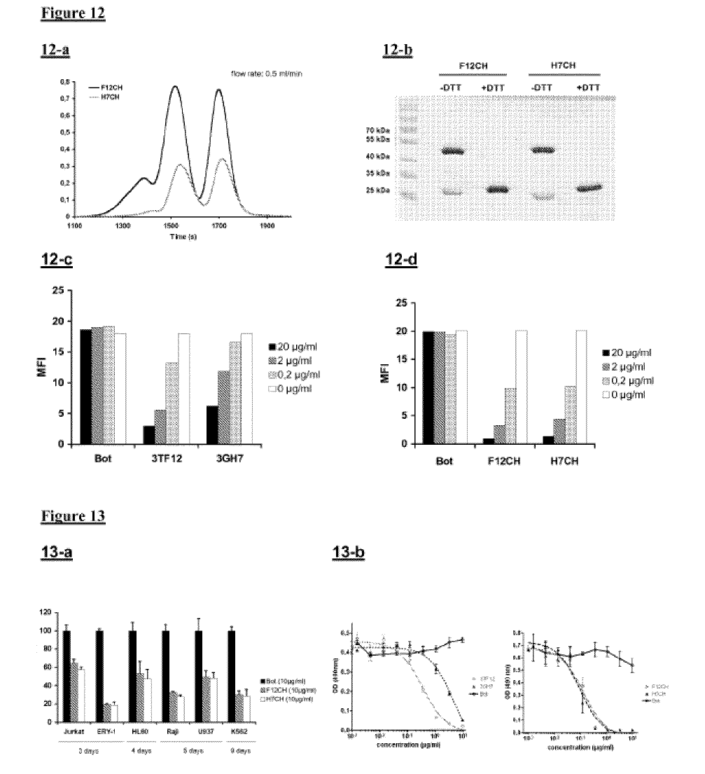

FIG. 12 (see FIGS. 12-a, 12-b, 12-c and 12-d) illustrate the results obtained during conversion of the monovalent format of the antibodies 3TF12 and 3 GH7 into the bivalent format F12CH and H7CH.

FIGS. 12-a and 12-b: Production of bivalent scFvCH antibodies (that is to say F12CH and H7CH) from the bacterial expression vector pSYN-HIS-CYS available from Bin Liu (UCSF). More particularly, FIG. 12-a shows the FPLC Superdex 75 chromatogram showing an elution profile by gel filtration of antibodies purified by Ni-NTA affinity chromatography. FIG. 12-b shows an SDS-PAGE gel analysis of dimeric antibody fractions isolated after gel filtration (fractions of about 55 kDa at the peak at 1500 seconds) under reducing (+DTT) or non-reducing (−DTT) conditions.

FIGS. 12-c and 12-d: Comparison of the dimeric and monomeric formats of antibodies for inhibiting the binding of holo-Tf to Raji cells. Raji cells are incubated with monovalent (FIG. 12-c) or bivalent (FIG. 12-d) antibodies for one hour at 4° C. at the concentrations indicated. 500 nM holo-Tf-FITC is then added to the cells for one hour at 4° C. After washing, the fluorescence is measured by FACS.

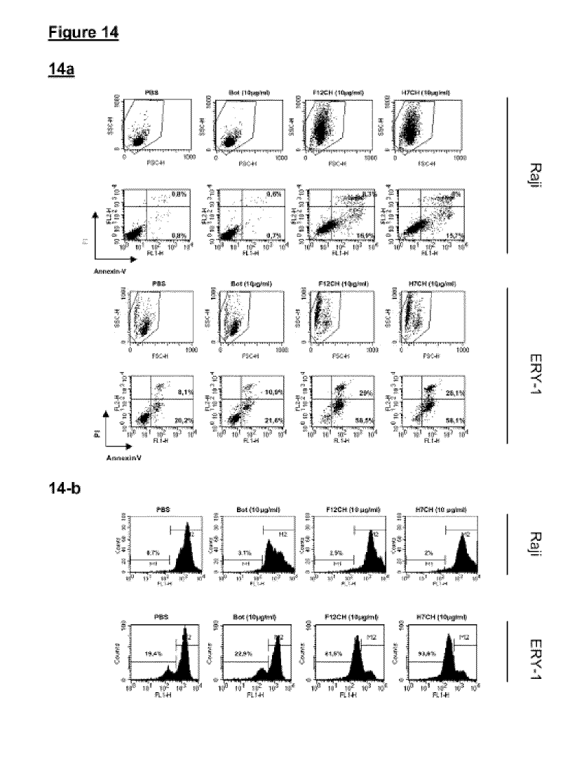

FIG. 13 illustrates the antiproliferative effect of the bivalent format.

FIG. 13-a: Several cell lines of haematopoietic origin are incubated for several days in the presence of bivalent soluble antibodies at 10 μg/ml. The cell viability is quantified with the aid of an MTT test (CellTiter 96 Aqueous One Solution Cell Proliferation Assay, Promega) (OD 490 nm).

FIG. 13-b: ERY-1 cells are incubated for three days with monovalent (on the left) or bivalent (on the right) soluble anti-TfR antibodies at the concentrations indicated. The viability is quantified by an MTT test.

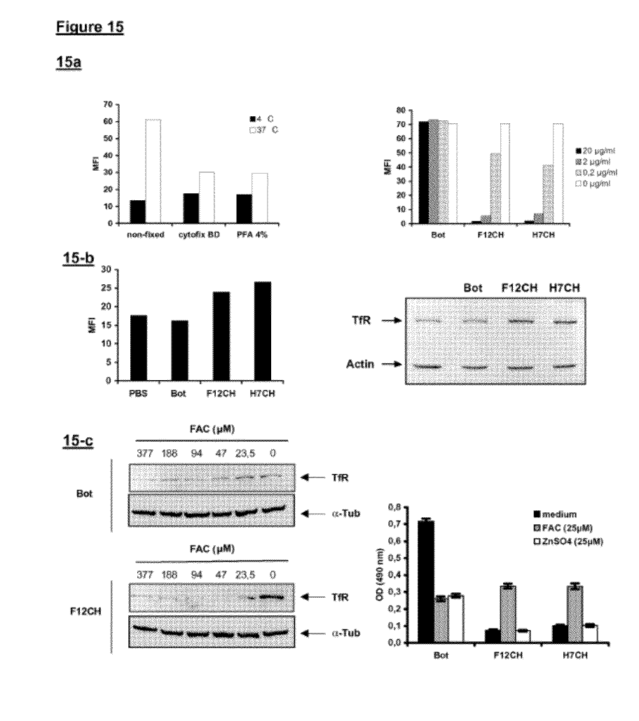

FIG. 14 illustrates the induction of the cell death of the Raji and ERY-1 lines by the anti-TfR F12CH and H7CH.

More particularly, FIG. 14-a illustrates the marking of the translocation of phosphatidylserines. Raji (top diagram) and ERY-1 (bottom diagram) lines are treated for 4 and 2 days respectively with the antibodies F12CH, H7CH and the negative control Bot at 10 μg/ml. The cells were washed and then marked with propidium iodide (PI) and annexin V-FITC. Positive annexin V, negative PI marking corresponds to apoptotic cells, while a positive annexin V, positive PI marking corresponds to necrotic cells.

FIG. 14-b illustrates the marking of the depolarization of the mitochondrial membrane. Raji (top diagram) and ERY-1 (bottom diagram) cell lines are treated in the same manner as in 14-a. After washing, the cells are marked with 3,3′-dihexyloxacarbocyanine (DIOC6). A drop in the intensity of the fluorescence correlates with a drop in the mitochondrial potential.

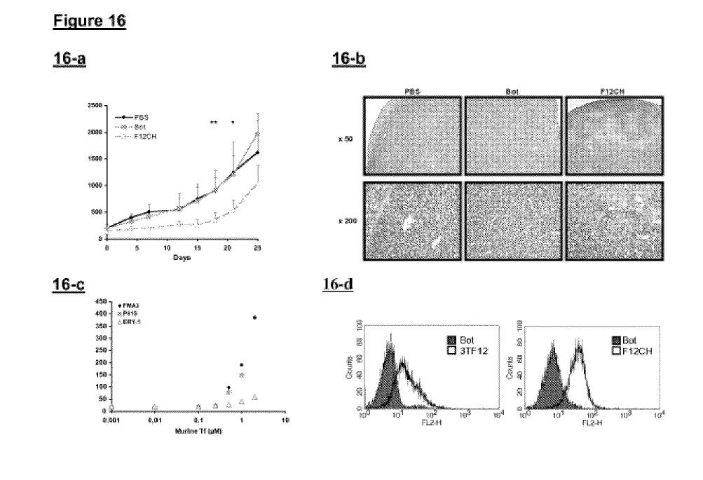

FIG. 15 illustrates the action mechanism of anti-TfR F12CH and H7CH.

In FIG. 15-a, on the left, Raji cells, fixed or non-fixed, are incubated in the presence of 500 nM Tf-FITC for three hours at 37° C. or 4° C. After washing, the fluorescence is measured by FACS.

FIG. 15-a on the right: Raji cells are incubated with antibodies at the concentrations indicated for one hour at 37° C., before being incubated in the presence of 500 nM Tf-FITC for 3 hours. After washing, the fluorescence is measured by FACS.

FIG. 15-b on the left: Raji cells are incubated for 4 days with antibodies at the concentration of 10 μg/ml. The cells are then washed in glycine buffer (glycine 50 mM, NaCl 500 mM, pH 2.8), before being fixed and marked by Tf-FITC (500 nM) for 3 hours at 37° C.

FIG. 15-b on the right: Raji cells are incubated for 4 days with antibodies at 10 μg/ml. Protein extracts are then obtained with the aid of a lysis buffer (Tris-HCl pH 7.5 50 mM, NaCl 250 mM, EDTA pH 8 10 mM, DTT 1 mM, NP40 1%, protease inhibitors (Roche)). After migration on SDS-PAGE gel, the proteins are transferred on to a nitrocellulose membrane. The expression of the TfR and actin (the latter serving as a control for the gel batch) is quantified with the aid of an anti-TfR murine antibody (Zymed) and an anti-alpha-actin goat antibody (Santa Cruz). Detection is achieved with the aid of two antibodies coupled to the enzyme HRP which recognize the murine immunoglobulins for the TfR and the goat immunoglobulins for the actin.

FIG. 15-c on the left: Raji cells are treated with F12CH or Bot antibodies at 10 μg/ml for 4 hours in the presence of ferric ammonium citrate (FAC) at the concentrations indicated. The cells are then washed and protein extracts are obtained in the same manner as above. After migration on SDS-PAGE gel and transfer on to a membrane, the TfR is quantified with the aid of an anti-TfR murine antibody (Zymed) and the alpha-tubulin (control of the gel batch) is quantified with the aid of an anti-alpha-tubulin murine antibody (Santa Cruz).

FIG. 15-c on the right: ERY-1 cells are treated with antibodies at 5 μg/ml in the presence of ferric ammonium citrate (FAC) or zinc sulfate (ZnSO₄) at 25 μM. The cell viability is quantified with the aid of an MTT test (CellTiter 96 Aqueous One Solution Cell Proliferation Assay, Promega).

FIG. 16 illustrates an in vivo study of the antibody F12CH.

FIG. 16-a: Athymic nude mice are irradiated at 4 Gy before subcutaneous injection of 2 million ERY-1 cells into each animal. Once the tumours have reached an average volume of 200 mm³, the mice are divided up into three cages each containing five mice. A volume of 200 μl of PBS or containing 200 μg of antibodies is then injected twice a week intraperitoneally into each mouse in the side opposite to the location of the tumour at D₀. The tumour growth is measured with the aid of a calliper in accordance with the formula: volume=(Π/6)×(length+width)³. The mice are sacrificed at D₂₅. The differences in the mean between the groups are analysed with the aid of a Mann-Whitney test (* for p<0.01, ** for p<0.005).

FIG. 16-b: After removal, the tumours are fixed and then marked with haematoxylin-eosin-saffron (HES).

FIG. 16-c: FMA3, P815 (P815 and FMA3 lines derived from a murine mastocytoma) and ERY-1 cells are incubated with biotinylated murine holotransferrin (Tfm) (Rockland) at the concentrations indicated for one hour at 4° C. The binding of the Tfm to the cells is detected with the aid of streptavidin coupled with phycoerythrin (BD Pharmingen). After washing, the fluorescence is measured by FACS.

FIG. 16-d left: FMA3 cells are incubated with the 3TF12 antibody (monomeric) or the Bot antibody at the concentration of 50 μg/ml for one hour at 4° C. The binding of the antibodies to the cells is measured with the aid of a secondary antibody directed against the myc tag (anti-c-myc, Sigma) of the 3TF12 and Bot antibodies and then a murine anti-immunoglobulin antibody coupled with phycoerythrin (BD Pharmingen). After washing, the fluorescence is measured by FACS. The curve shaded grey corresponds to the fluorescence of the Bot marking, while the transparent curve corresponds to that of the 3TF12 marking.

FIG. 16-d right: FMA3 cells are incubated with the F12CH antibody (dimeric) or the Bot antibody at the concentration of 50 μg/ml for one hour at 4° C. The binding of the antibodies to the cells is measured with the aid of a secondary antibody directed against the histidine tag (penta-His antibody, Qiagen) of the F12CH and Bot antibodies and then a murine anti-immunoglobulin antibody coupled with phycoerythrin (BD Pharmingen). After washing, the fluorescence is measured by FACS. The curve shaded grey corresponds to the fluorescence of the Bot marking, while the transparent curve corresponds to that of the F12CH marking.

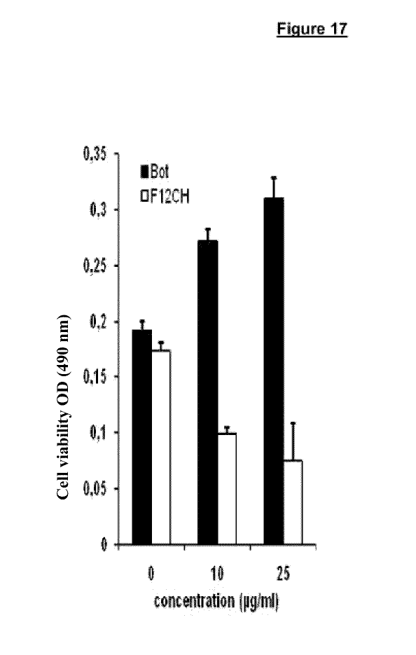

FIG. 17 shows a proliferation test on human blood mononuclear cells (PBMC) treated with F12CH. The cells are seeded at 2.10⁴ cells per well in RPMI medium, 10% SVF supplemented with IL-2 (50 ng/ml). The cells are treated with Bot or F12CH antibodies (dimeric format produced in bacteria) for 3 days at the concentrations indicated. The viability is measured with the aid of an MTT test (CellTiter 96 Aqueous One Solution Cell Proliferation Assay, Promega) (OD 490 nm).

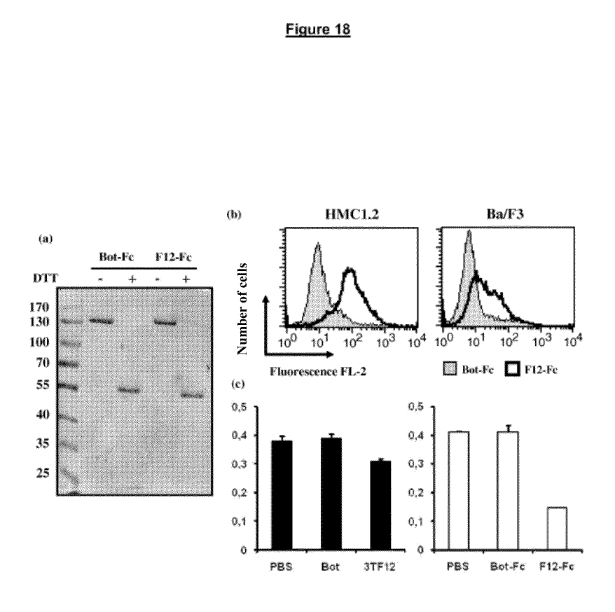

FIG. 18 shows the study of the scFv-Fc format of Bot-Fc and F12-Fc antibodies.

FIG. 18 (a): SDS-PAGE gel analysis of the purification of the Bot-Fc and F12-Fc antibodies under reducing and non-reducing conditions. One μg of antibodies was deposited in each well of the gel.

FIG. 18 (b): Membrane marking of human HMC1.2 (left) and murine Ba/F3 (right) cells by Bot-Fc (grey) and F12-Fc (black) antibodies. The cells were incubated with the antibodies at 10 μg/ml for 1 hour at 4° C. The binding of the scFv-Fc to the cells was detected with the aid of a secondary antibody directed against the human Fc fragment and coupled with phycoerythrin (Rockland).

FIG. 18 (c): Tests of the effect of the two formats of scFv versus scFv-Fc antibodies on the proliferation of the HMC1.2 line. The HMC1.2 cells (10⁴ per well, in sixplicate) were seeded in RPMI medium, 10% SVF with the antibodies at 10 μg/ml (either in the scFv format or in the scFv-Fc format). The cell viability was measured at the end of 6 days (scFv, left diagram) and 4 days (right diagram) by an MTT test (CellTiter 96 Aqueous One Solution Cell Proliferation Assay, Promega), (OD 490 nm).

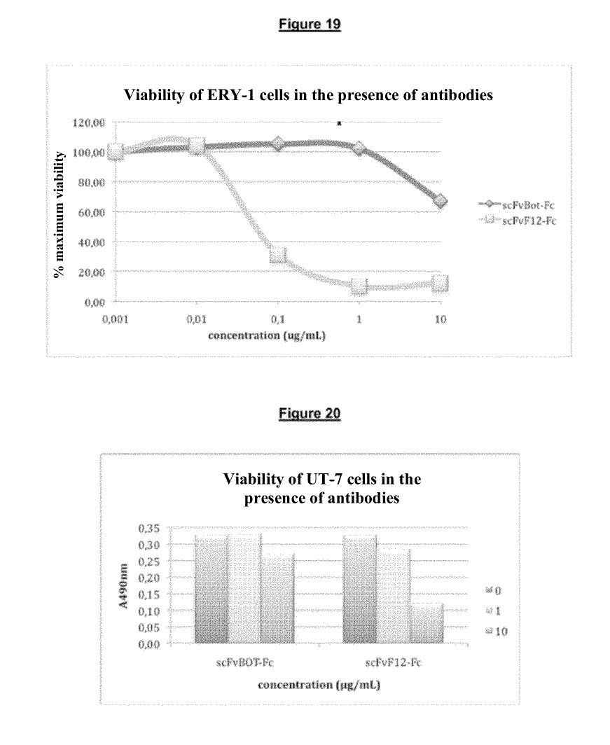

FIG. 19 shows the evaluation of the sensitivity of ERY-1 cells in the bivalent scFv-Fc format for the F12 antibody. A 96-well plate was seeded with 4,000 cells per well in sixplicate in RPMI/SVF 10% medium containing 0.001, 0.01, 0.1, 1 or 10 μg/ml of soluble antibodies. The cells were incubated in a humidity chamber with 5% CO₂ at 37° C. for 4 days. The number of viable cells was quantified with the aid of an MTT test (CellTiter 96 Aqueous One Solution Cell Proliferation Assay, Promega). The results are shown as the % viability in relation to a control without antibodies.

FIG. 20 shows the inhibition of the proliferation of the UT-7 line in the presence of 3TF12 antibodies. A 96-well plate was seeded with 4,000 cells per well in sixplicate in the medium IMDM/SVF 10%/erythropoietin at 2 U/ml containing 0, 1 or 10 μg/ml of soluble antibodies. The cells were incubated in a humidity chamber with 5% CO₂ at 37° C. for 4 days. The number of viable cells was quantified with the aid of an MTT test (CellTiter 96 Aqueous One Solution Cell Proliferation Assay, Promega). The results show the value of the absorbance at 490 nm as a function of the antibody concentration applied.

The examples below illustrate the invention without in any way limiting it.

### EXAMPLE 1

### Six Different Anti-TfR Antibodies Produced by a Functional Selection

The anti-TfR antibodies were obtained in vitro by phage display in an scFv format (single chain fragment variable format) and were selected for their capacity for being endocyted (internalized) specifically inside mammary tumour cells (SKBR3).

This selection enabled 6 antibodies each of molecular weight 28 kDa to be obtained, called respectively 3TF12 (SEQ ID NO: 49), 3TF2 (SEQ ID NO: 50), 3 GH9 (SEQ ID NO: 51), C3.2 (SEQ ID NO: 52), 3TG9 (SEQ ID NO: 53) and 3 GH7 (SEQ ID NO: 54).

These single chain fragment variable (scFv) are also called monovalent fragments and represent the monomeric form.

The antibodies of the invention are specific for the TfR: the binding of each anti-TfR scFv phage is inhibited in a dose-dependent manner by the presence of holotransferrin (FIG. 7). They recognize close or common epitopes: the binding of each anti-TfR scFv phage is inhibited by the presence of a soluble anti-TfR scFv (FIG. 8). They overlap the binding site of holotransferrin (holo-Tf) to the TfR: each anti-TfR scFv inhibits the binding of holo-Tf to the TfR in a more or less pronounced manner with a maximum inhibition for 3TF12 and 3 GH7 (FIG. 9).

### EXAMPLE II

### Antiproliferative Effects of the Anti-TfR Antibodies and a Study of their Action Mechanism

Cancer cells of haematopoietic origin have an increased need for iron. A proliferation test was carried out on the Jurkat line developed from a T-acute lymphoblastic leukaemia for 3 days in the presence of the six anti-TfR scFvs.

Only the 3TF12 and 3 GH7 antibodies have a weak but reproducible antiproliferative activity (FIG. 10) on the Jurkat line.

The antibodies which have an antiproliferative effect on the Jurkat line, that is to say 3TF12 and 3 GH7, are also those which best inhibit the binding of Tf to the TfR.

In order to determine whether this antiproliferative effect is a characteristic of the antibodies or a characteristic of the origin of the cell line used, the viability of five other cancer lines of haematopoietic origin was tested over several days in the presence of the 3TF12 or 3 GH7 antibodies at 10 μg/ml (FIG. 11).

Antiproliferative marking effects are observed (up to 80% inhibition on the Raji and ERY-1 lines), depending on the cell type tested.

The change from the monomeric format of the antibodies to the dimeric format (F12CH for 3TF12 and H7CH for 3 GH7) enabled the affinity of the antibodies for the TfR to be increased: the bivalent format of the antibodies improves the inhibition of Tf on the TfR (FIGS. 12-a to 12-d). The antiproliferative effect is improved.

The molecular weight of dimeric or bivalent fragment variables is about 55 kDa.

This change in format enables the antiproliferative effect of monovalent scFvs to be potentiated (FIG. 13) on all the lines testes, as well as the IC₅₀ relative to each antibody to be reduced (Table 1).

TABLE 1
Comparison of the IC₅₀ obtained for each antibody by a proliferation
test on the line ERY-1 incubated in the presence of the antibody,
in the monomeric or dimeric format, for three days.

| Antibody | IC₅₀ (μg/ml) |
|---|---|
| Monomers | |
| 3TF12 | 0.35 |
| 3GH7 | 4.86 |
| Dimers | |
| F12CH | 0.10 |
| H7CH | 0.09 |

The bivalent format of the antibody improves the antiproliferative effect of anti-TfR 3 GH7 and 3TF12. This effect is observed on all the lines tested (FIG. 13).

Furthermore, the anti-TfR F12CH and H7CH induce cell death by apoptosis (for the ERY-I line) or by autophagy (for the Raji line) (FIG. 14).

In fact, the anti-TfR induce the triggering of cell death of the apoptotic type passing through the mitochondrial route for the ERY-1 line. As regards the Raji line, after treatment a translocation of phosphatidylserine is observed, without a drop in the mitochondrial potential. Furthermore, the granularity of the treated cells increases (FIG. 14-a top), which would indicate that this phenomenon of cell death is associated with an autophagy process for this line.

The inventors of the present invention then discovered the action mechanism responsible for the cytotoxicity of these antibodies.

Given that the antibodies inhibit the binding of Tf to the TfR at 4° C., the inventors wanted to ascertain whether this inhibition is maintained at 37° C. In this way it is possible to know if the internalization of holo-Tf is blocked by the bivalent anti-TfR in a dynamic context.

The Tf-FITC associated with the cells was first quantified after three hours of incubation at 37° C. (FIG. 15-a on the left). In order to distinguish the signal relating to the internalized Tf-FITC from the signal of the Tf-FITC simply bound on the surface, the same experiment was repeated on cells incubated beforehand in a fixing solution (paraformaldehyde 4% prepared extemporaneously or commercial fixing solution, BD). A reduction in the signal of 50% is observed if the cells are fixed, with respect to non-fixed cells. A part of the fluorescence associated with the cells therefore corresponds to an intracellular location. When cells are incubated beforehand with F12CH or H7CH at various concentrations (0.2 to 20 μg/ml), a dose-dependent inhibition of the signal is observed.

This result gives rise to the conclusion that the antibodies F12CH and H7CH effectively block the internalization of holo-Tf in the cells.

The inventors then wanted to ascertain whether the fixing of the antibodies F12CH and H7CH on cancer cells causes a modification of the level of expression of the TfR.

For this, Raji cells are incubated for 4 days with the anti-TfR F12CH or H7CH at 10 μg/ml. The cells are then washed in glycine buffer (pH 2) to eliminate the scFv antibodies bound to the cell surface, and then incubated with Tf-FITC to quantify the membrane expression of the TfR (FIG. 15-b on the left) at the cell surface.

It is found that the treated cells overexpress the TfR. If protein extracts are obtained from the same cells and the expression of the TfR is analysed by western blotting (FIG. 15-b on the right), this overexpression is also found in the treated extracts compared with the control extracts. There is therefore a global increase in the expression of the TfR.

The expression of the TfR is regulated by the presence of IRE (iron responsive elements) motifs present on the mRNA of TfR, which stabilize it if the concentration of iron in the cells is low. Conversely, in the case of excess iron, the mRNA of the TfR is degraded and the TfR is no longer synthesized (Daniels et al., The transferrin receptor part I or part II, Clin Immunol., 2006).

For this reason, the inventors wanted to ascertain whether the overexpression of the TfR observed following the action of the antibodies F12CH or H7CH is linked with a depletion in iron induced by the antibodies or associated with another mechanism.

For this, the Raji cells are treated with the control antibody Bot and the F12CH antibody for 4 days in the presence of soluble iron (FAC, ferric ammonium citrate) in the culture medium at various concentrations (FIG. 15-c on the left).

It is found that the presence of iron suppresses the expression of the TfR in both cases (Bot and F12CH), which proves that the overexpression of the TfR observed for F12CH is certainly linked to a depletion of iron induced by blocking of the endocytosis of transferrin.

Finally, the inventors wanted to ascertain whether the cytotoxicity of the antibodies is linked to the depletion of iron which they induce. The most sensitive cell line (ERY-1) is treated for 3 days in the presence of the antibodies supplemented by metal cations (iron or zinc, the latter used as a negative control) and the cell viability is quantified with the aid of an MTT test (FIG. 15-c on the right). It is found that an exogenous supply of soluble iron cancels the cytotoxicity of the antibodies.

In conclusion, the overexpression of the TfR by the cells observed following the action of F12CH is certainly linked to a depletion of iron caused by F12CH.

This mechanistic property is unique in comparison with the mode of action of other anti-TfR murine antibodies described previously. In fact, in contrast to the antibodies of the invention, the latter require a multivalent format to perform their cytotoxic action, which correlates with a reduction in the expression of the TfR at the membrane level (IgG A24, (Lepelletier et al., Cancer Res 67: 1145, 2007; Moura et al., Blood 103: 1838, 2004), IgA 42/6 (Taetle et al., Cancer Res 46, 1759-63, 1986), “avidin-fused lgG3” (Ng et al., Blood 108, 2745-54, 2006), IgM R17 208 (Lesley and Schutle, Mol Cell Biol 5, 1814-21, 1985)).

The inventors have thus been able to ascertain that the antibodies of the invention induce a depletion of iron in cells, which results in an increase in the iron of the receptor (TfR) at the surface of the cell.

However, this information is not sufficient per se to ascertain on what cell lines the antibodies of the invention are going to be active, since it is impossible to ascertain whether a cell line is sensitive to a deprivation of iron. In fact, the reasons for the greater or lesser sensitivity to a deprivation of iron are not known, and in particular do not depend on the level of the receptor on the surface (see FIG. 2C of document D3 mentioned above).

### EXAMPLE III

### Study of a Murine Model of a Xenografted Human Tumour

It has been demonstrated in the above examples that the antibodies F12CH and H7CH have a pronounced anticancer activity on several cell lines of haematopoietic origin in vitro.

In order to ascertain whether this anticancer activity of the antibody F12CH is maintained in a tumour environment in vivo, a model of a subcutaneous xenograft was developed in the nude mouse from the cell line ERY-1. Once the graft had taken effect (average tumour volume 200 mm³), 200 μg of the antibody F12CH or Bot were injected into each animal twice a week.

The tumour growth was measured over 25 days and is shown on FIG. 16-a.

It is found that the antibody F12CH slows down tumour growth in comparison with the control carrier (injection of PBS buffer alone) and the isotype control (injection of a non-specific antibody produced under the same conditions).

In the tumour sections of treated mice (FIG. 16-b), the presence of fibrosis and prenecrotic cells can be seen.

The effect observed is all the more physiologically relevant since it has been demonstrated that the murine holo-Tf present in the host at concentrations of the order of 10 mM has an increased reactivity for human TfR (FIG. 16-c) and that the antibody F12CH has an increased reactivity with the murine TfR (FIG. 16-d) present on certain cells of the host.

The animal model used to test these antibodies thus reflects some of the possible interactions which would occur during administration of the antibody F12CH in man.

It is important to note that in the format used, the antibody F12CH is deprived of the fragment Fc and therefore cannot enlist effectors of the immune system to cause an antitumour response of the ADCC (antibody-dependent cell-mediated cytotoxicity) or CDC (complement-dependent cytotoxicity) type, which demonstrates that the effect observed is characteristic of the mode of action of the antibody.

Their particular mode of cytotoxicity, that is to say cytotoxicity independent of the natural effector functions of antibodies of the ADCC or CDC type and associated with an increase in the TfR on the surface of target cells, thus renders the antibodies of the invention particularly advantageous as therapeutic antibodies with a high potential for treatment of cancers.

### EXAMPLE IV

### Effect of the Antibodies on the Proliferation of Activated Blood Mononuclear Cells

The antibodies 3TF12 and 3 GH7 in the bivalent format are tested for autoimmune diseases or also for transplants, as preventive therapy against rejection of the graft.

The expression of TfR/CD71 on the surface of T cells plays an essential role the activation and maintenance of the proliferation of T lymphocytes. The anti-TfR inhibitor antibodies could be used as immunosuppressants in autoimmune diseases or in the prevention of rejection of a graft or of the reaction of the graft to the host.

The effect of the antibodies 3TF12 and 3 GH7 in the scFvCH format (bivalent, 50 kD, produced in bacteria) was tested on the proliferation of human blood mononuclear cells activated by the cytokine IL-2 (“PBMC”).

Preparations, realized under the same conditions, of the antibodies 3TF12 (anti-TfR) (F12CH) and Bot (non-specific) were tested (see FIG. 17).

A dose-dependent inhibition of the proliferation of the activated PBMC is observed in the presence of the anti-TfR antibody F12.

It is to be noted that the antibodies are produced in bacteria and that traces of lipopolysaccharide (LPS) in this purification may explain the stimulation of the proliferation of the PBMC in the presence of the non-specific antibody Bot.

The antibody F12, in the dimeric format scFvCH, thus inhibits the IL-2-dependent proliferation (50% inhibition at the concentration of 10 μg/ml) of peripheral lymphocytes and could thus be used as an immunosuppressant.

### EXAMPLE V

### Production of Antibodies in the scFv-Fc Format and the Effect on Various Cell Lines

The bivalent format used by the inventors (scFv-CH) allowed a significant improvement in the antiproliferative effects of the antibodies, initially in a monovalent format of 3TF12 and 3 GH7. However, even though this novel format has a higher molecular weight that the standard scFv format (55 versus 28 kD, greater than the renal filtration limit), its half-life in serum is only a few hours less than that of complete antibodies of the IgG isotype (half-life 21 to 24 days in man for the isotypes IgG1, 2 and 3). This low molecular weight associated with the absence of the fragment Fc could considerably limit the serum stability and in vivo efficacy of the antibodies (even though an effect on the xenograft model in the mouse is observed). Furthermore, the presence of a fragment Fc would allow, in addition to binding to the TfR, on the one hand enlisting of the first component of the complement cascade, C1q, and initiation of the complement cascade, and on the other hand binding to Fc receptors on “natural killer” (NK) cells and macrophages for mediation of ADCC effector functions. By a mechanism independent of the depletion of iron, this could increase the cytotoxic activity of the antibody on the cells which express high levels of the TfR on their surface.

The antibodies H7 and F12 are prepared in the 110 kD scFv-Fc format, which indicates that an scFv fragment is fused to the C-terminal end of the constant CH2 and CH3 domains of a human IgG1. Such proteins homodimerize naturally to form a bivalent molecule of about 110 kDa (2×55 kDa).

To obtain antibodies in the scFv-Fc format, the VH and VL sequences of the scFv of Bot, 3TF12 and 3 GH7 are subcloned into the vector pFuse-hG1 (described in Moutel, S. et al., 2009. A multi-Fc-species system for recombinant antibody production. BMC. Biotechnol. 9, 14), enabling the constructions pFuse-hG1-Bot-Fc, pFuse-hG1-F12-Fc and pFuse-hG1-H7-Fc to be obtained. The expression of this antibody format is effected by transfection of CHO cells. The cells will then secrete the scFv-Fc in the medium over 3 days. The antibodies are then purified over protein A-agarose resin and then concentrated.

FIG. 18 (a) illustrates the production of the antibodies F12 and Bot in the scFv-Fc format. Under non-reducing conditions, the dimeric format of the antibodies at about 110 kD is detected. By addition of DTT, the disulfide bridges are reduced and the monomeric forms migrate at 55 kD, as expected.

FIG. 18 (b) shows that the antibody F12 in the format scFv-Fc binds to the HMC1.2 cells (human mastocytoma line) and to murine BaF/3 cells (murine pro-B haematopoietic line). As a control, it is shown that there is no binding of an antibody directed against botulinum toxin (Bot) to the HMC1.2 cells.

FIG. 18 (c) shows that the proliferation of HCM1.2 cells is inhibited by the antibody scFv-F12-Fc with a higher intensity that with the scFv-F12 format at the same concentration. The two formats of the non-specific antibody (Bot) have no effect.

The same result of growth inhibition was obtained with the antibody H7 (not shown here).

FIG. 19 shows that on the ERY-1 cells, the IC_{H} is less than 0.1 μg/ml of the F12 antibody. The scFv-Fc format is therefore just as effective (and indeed more effective) as the bivalent format scFvCH (FIG. 13-b).

The antibody F12 in the bivalent format scFv-Fc was tested on the line UT-7, a human erythroleukaemic line. A potent inhibitor effect (70% inhibition) of the antibody F12 is observed at the concentration of 10 μg/ml (FIG. 20).

### GENERAL CONCLUSION

Six different antibodies specific for the transferrin receptor which recognize neighbouring epitopes on the TfR and are capable of inhibiting the natural ligand of the TfR, holotransferrin, were selected, by presentation of the antibodies on the surface of phages, for their capacity for being endocyted by living cancer cells, by endocytosis mediated by receptors. This functional selection allowed six fragments of human anti-TfR monovalent antibodies (anti-TfR scFv) to be obtained, that is to say single chain fragment variables (scFv) of molecular weight about 28 kD.

The six anti-TfR scFv antibody fragments of the functional ligand type obtained in this selection all interfere with the natural ligand holotransferrin (holo-Tf), which binds to the TfR, and therefore all disrupt the binding of holo-Tf to the TfR.

The anti-TfR scFv fragments which have the highest affinity for the TfR, that is to say 3TF12 (SEQ ID NO: 49) and 3 GH7 (SEQ ID NO: 54), inhibit the proliferation of various haematopoietic cancer cell lines and have particularly interesting anticancer properties.

The inhibition potential on the proliferation of cells of the antibodies 3TF12 and 3 GH7 was improved by developing bivalent antibody formats of 55 kD having an improved affinity, that is to say F12CH and H7CH, due to the bivalent nature.

This change in format (from monovalent to bivalent) allowed an improvement in the IC₅₀ relative to each antibody.

The cytotoxicity mechanism of these high-affinity endocytable antibodies is different to that of the anti-TfR murine growth inhibitor monoclonal antibodies described previously in the literature.

In fact, F12CH and H7CH induce an increase in the number of TfR on the surface of cells (instead of reducing it by increasing their degradation), while considerably inhibiting the cell absorption of holo-Tf and while inducing cell death by apoptosis and/or autophagy.

These properties have enabled a novel family of completely human anti-TfR antibody fragments to be defined which are suitable for immunotherapy of iron-dependent tumours for durable proliferation and which express a large number of TfR.

The fact that the antibodies of the invention are produced from a bank of human antibodies is very advantageous, since they therefore do not require a humanization stage for their transfer to preclinical development.

The six antibodies of the invention (whether in their monovalent or bivalent format) are capable of targeting slightly different epitopes (each epitope sharing motifs with the binding site of the natural ligand Tf) and have different affinities for their target.

An alternative clinical use of one or other of these antibodies could prove to be particularly advantageous, since it would allow resistance phenomena induced by the treatment to be avoided.

The antibodies of the invention function by a unique mechanism which has yet never been described to date. They induce cell death of cancer cells by depriving them of iron without the need for a multivalent format agglutinating the TfR on the cell surface.

Furthermore, an incubation of a few days in vitro in the presence of the antibodies causes an increase in the TfR on the cell surface. If this observation is reproduced in vivo, this unique property will increase the “visibility” of the cells to be targeted.

The antibody format used in vivo is that of a bivalent dimeric scFv of about 55 kDa.

In order to increase the antitumour properties of the antibodies of the invention (in both their monovalent and bivalent format), a modification of the format of the antibody by addition of an Fc fragment to obtain a complete antibody or by pegylation, which would allow an increase in the half-life of the antibodies, may be envisaged. An scFv-Fc format (110 kDa) where the scFv is fused to the Fc region of a human immunoglobulin of the IgG1 type has been produced for the scFv antibodies F12 and H7. This format reproduces the cytotoxic effects of the antibodies in the scFvCH format in vitro. The presence of Fc regions could additionally allow the enlisting of effectors of the immune system and increase the antitumour effect of the antibody.

The antibodies of the invention (in both their monovalent and bivalent format), due to their specificity for the TfR, their particular mode of cytotoxicity and their low immunogenicity combined with their human origin, therefore prove to be therapeutic molecules with a high potential for the treatment of cancer.

1. Molecular construction, characterized in that it comprises at least one amino acid sequence chosen from:
SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36,
the said amino acid sequence corresponding to a region determining complementarity with the antigen (“CDR”, complementarity determining region) of the variable domain of the heavy chain (CDR-H) or of the light chain (CDR-L) of an antibody which targets the human transferrin receptor (TfR).

2. Molecular construction according to claim 1, characterized in that it is made up of at least one of the amino acid sequences chosen from: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 and SEQ ID NO: 36.

3. Molecular construction according to claim 1, characterized in that at least one of the sequences SEQ ID NO: 1 to SEQ ID NO: 36 is included in one of the amino acid sequences chosen from:
SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48,
each of the said sequences SEQ ID NO: 37 to SEQ ID NO: 48 corresponding to the variable domain of the heavy chain or of the light chain of an antibody which targets the human transferrin receptor (TfR),
the said sequence SEQ ID NO: 37 comprising the sequences SEQ ID NO: 1, SEQ ID NO: 2 and SEQ ID NO: 3,
the said sequence SEQ ID NO: 38 comprising the sequences SEQ ID NO: 4, SEQ ID NO: 5 and SEQ ID NO: 6,
the said sequence SEQ ID NO: 39 comprising the sequences SEQ ID NO: 7, SEQ ID NO: 8 and SEQ ID NO: 9,
the said sequence SEQ ID NO: 40 comprising the sequences SEQ ID NO: 10, SEQ ID NO: 11 and SEQ ID NO: 12,
the said sequence SEQ ID NO: 41 comprising the sequences SEQ ID NO: 13, SEQ ID NO: 14 and SEQ ID NO: 15,
the said sequence SEQ ID NO: 42 comprising the sequences SEQ ID NO: 16, SEQ ID NO: 17 and SEQ ID NO: 18,
the said sequence SEQ ID NO: 43 comprising the sequences SEQ ID NO: 19, SEQ ID NO: 20 and SEQ ID NO: 21,
the said sequence SEQ ID NO: 44 comprising the sequences SEQ ID NO: 22, SEQ ID NO: 23 and SEQ ID NO: 24,
the said sequence SEQ ID NO: 45 comprising the sequences SEQ ID NO: 25, SEQ ID NO: 26 and SEQ ID NO: 27,
the said sequence SEQ ID NO: 46 comprising the sequences SEQ ID NO: 28, SEQ ID NO: 29 and SEQ ID NO: 30,
the said sequence SEQ ID NO: 47 comprising the sequences SEQ ID NO: 31, SEQ ID NO: 32 and SEQ ID NO: 33,
the said sequence SEQ ID NO: 48 comprising the sequences SEQ ID NO: 34, SEQ ID NO: 35 and SEQ ID NO: 36.

4. Molecular construction according to claim 3, characterized in that at least one of the sequences SEQ ID NO: 37 to SEQ ID NO: 48 is included in one of the amino acid sequences chosen from:
SEQ ID NO: 49, SEQ ID NO: 50, SEQ ID NO: 51, SEQ ID NO: 52, SEQ ID NO: 53, SEQ ID NO: 54,
each of the said sequences SEQ ID NO: 49 to SEQ ID NO: 54 comprising the variable domain of the heavy chain and of the light chain of an antibody which targets the human transferrin receptor (TfR),
the said sequence SEQ ID NO: 49 comprising the sequences SEQ ID NO: 37 and SEQ ID NO: 38 linked to one another by the peptide linker having an amino acid sequence SEQ ID NO: 109,
the said sequence SEQ ID NO: 50 comprising the sequences SEQ ID NO: 39 and SEQ ID NO: 40 linked to one another by the peptide linker SEQ ID NO: 109,
the said sequence SEQ ID NO: 51 comprising the sequences SEQ ID NO: 41 and SEQ ID NO: 42 linked to one another by the peptide linker SEQ ID NO: 109,
the said sequence SEQ ID NO: 52 comprising the sequences SEQ ID NO: 43 and SEQ ID NO: 44 linked to one another by the peptide linker SEQ ID NO: 109,
the said sequence SEQ ID NO: 53 comprising the sequences SEQ ID NO: 45 and SEQ ID NO: 46 linked to one another by the peptide linker SEQ ID NO: 109,
the said sequence SEQ ID NO: 54 comprising the sequences SEQ ID NO: 47 and SEQ ID NO: 48 linked to one another by the peptide linker SEQ ID NO: 109.

5. Molecular construction according to claim 4, characterized in that it is in the form of a monomer.

6. Molecular construction according to claim 4, characterized in that it is in the form of a dimer.

7. The molecular construction of claim 1 further comprising a fragment Fc.

8. The molecular construction of claim 1 further characterized as an antibody or an antibody fragment.

9. Nucleotide sequences, characterized in that they code respectively for the amino acid sequences SEQ ID NO: 1 to SEQ ID NO: 36 as defined in claim 1, and in that they are represented respectively by the sequences SEQ ID NO: 55 to SEQ ID NO: 90.

10. Nucleotide sequences, characterized in that they code respectively for the amino acid sequences SEQ ID NO: 37 to SEQ ID NO: 48 as defined in claim 3, and in that they are represented respectively by the sequences SEQ ID NO: 91 to SEQ ID NO: 102.

11. Nucleotide sequences, characterized in that they code respectively for the amino acid sequences SEQ ID NO: 49 to SEQ ID NO: 54 as defined in claim 4, and in that they are represented respectively by the sequences SEQ ID NO: 103 to SEQ ID NO: 108,
the said SEQ ID NO: 103 comprising the sequences SEQ ID NO: 91 and SEQ ID NO: 92 linked to one another by the peptide linker having a nucleotide sequence SEQ ID NO: 110,
the said SEQ ID NO: 104 comprising the sequences SEQ ID NO: 93 and SEQ ID NO: 94 linked to one another by SEQ ID NO: 110,
the said SEQ ID NO: 105 comprising the sequences SEQ ID NO: 95 and SEQ ID NO: 96 linked to one another by SEQ ID NO: 110,
the said SEQ ID NO: 106 comprising the sequences SEQ ID NO: 97 and SEQ ID NO: 98 linked to one another by SEQ ID NO: 110,
the said SEQ ID NO: 107 comprising the sequences SEQ ID NO: 99 and SEQ ID NO: 100 linked to one another by SEQ ID NO: 110, the said SEQ ID NO: 108 comprising the sequences SEQ ID NO: 10 and SEQ ID NO: 102 linked to one another by SEQ ID NO: 110.

12. Isolated nucleic acid molecule, characterized in that it comprises at least one of the nucleotide sequences SEQ ID NO: 55 to SEQ ID NO: 108 as defined in claim 9.

13. Expression vector, characterized in that it comprises a nucleic acid molecule as defined in claim 12.

14. Host cell or organism, characterized in that it comprises an expression vector as defined in claim 13.

15. Pharmaceutical composition comprising a therapeutically effective amount of the molecular construction of claim 1 in combination with a pharmaceutically acceptable carrier.

16. Composition according to claim 15, in which the molecular construction is used to vectorize one (or more) biologically active molecule(s).

17. Composition according to claim 15, in which the molecular construction is used for targeting liposomes and/or nanoparticles charged with one (or more) cytotoxic agent(s) and/or one (or more) agent(s) with a diagnostic aim.

18. Composition according to claim 15 for use as a medicament in the treatment of pathologies with an overexpression of the TfR.

19. Composition according to claim 18, in which the pathology is a cancer.

20. (canceled)

21. Composition according to claim 18, in which the pathology is an autoimmune pathology.
